(19) United States
(12) Patent Application Publication
(10) Pub. No.: US 2025/0130233 A1
(43) Pub. Date: Apr. 24, 2025

(54) METHODS FOR ENHANCED BCMA IMMUNOHISTOCHEMISTRY DETECTION IN HUMAN AND MONKEY TISSUE

(71) Applicant: Janssen Biotech, Inc., Horsham, PA (US)

(72) Inventors: Vinicius CARREIRA, San Diego, CA (US); Mathieu MARELLA, San Diego, CA (US); Ingrid CORNAX, San Diego, CA (US)

(21) Appl. No.: 18/705,252

(22) PCT Filed: Oct. 26, 2022
(86) PCT No.: PCT/US2022/078683

§ 371 Date: Apr. 26, 2024

Related U.S. Application Data
(60) Provisional application No. 63/272,600, filed on Oct. 27, 2021.

(51) Int. Cl. G01N 33/573; A61K 35/17; A61K 40/11; A61K 40/31; A61K 40/42; C07K 16/28; G01N 1/28; G01N 1/34; G01N 1/36; G01N 1/44; G01N 33/68

(52) U.S. Cl. CPC G01N 33/573; A61K 35/17; A61K 40/11; A61K 40/31; A61K 40/4215; C07K 16/2878; G01N 1/34; G01N 1/36; G01N 1/44; G01N 33/6863; G01N 2001/2873; G01N 2333/70578; G01N 2474/20

(57) ABSTRACT

Provided herein are methods of detecting the presence of a molecule in a sample, such as a bodily fluid or tissue of a patient.

### CROSS-REFERENCE TO RELATED APPLICATIONS

This application claims the benefit of priority to U.S. Ser. No. 63/272,600 filed Oct. 27, 2021, the contents of which is herein incorporated by reference in its entirety.

### FIELD

Provided herein are methods of detecting the presence of a molecule in a sample, such as a bodily fluid or tissue of a patient.

### SUMMARY

In one aspect, provided herein is a method of detecting a molecule of B cell maturation antigen (“BCMA”) in a formalin-fixed paraffin-embedded (“FFPE”) sample, comprising: sectioning and mounting of the FFPE samples; deparaffinizing the samples; heat-mediated antigen retrieval of the samples; pretreatment of the samples with blocking agents; contacting the sample with a first detection agent, wherein optionally the first detection agent comprises an antibody or antigen binding fragment thereof, that binds at least one molecule of BCMA in the sample; optionally, removing unbound sample; contacting the sample bound to the first detection agent with a second detection agent, wherein optionally the second detection agent is an antibody or fragment thereof; optionally removing the unbound second detection agent; and detecting the presence of the second detection agent bound to the sample; wherein detection above background of an amount of the second detection agent bound to the sample indicates the presence of at least one molecule of BCMA in the sample.

In some embodiments, the heat-mediated antigen retrieval is performed using an EDTA-based solution.

In some embodiments, the EDTA-based solution has a pH between 8.5 to 9.5. In some embodiments, the EDTA-based solution has a pH of 9.0.

In some embodiments, the step of heat-mediated antigen retrieval is performed for 5 minutes, 10 minutes, 15 minutes, 20 minutes, 25 minutes, 30 minutes or 60 minutes.

In some embodiments, the step of heat-mediated antigen retrieval is performed at a temperature between 85° C. and 100° C. In some embodiments, the step of heat-mediated antigen retrieval is performed at 100° C.

In some embodiments, the blocking agent blocks endogenous peroxidase. In some embodiments, the blocking agent is a peroxide block.

In some embodiments, the step of pretreating with peroxide block is performed for 2 minutes, 5 minutes, 8 minutes, 10 minutes or 15 minutes.

In some embodiments, the blocking agent blocks non-specific antibody binding. In some embodiments, the blocking agent is a Dako/Agilent Protein Block.

In some embodiments, the step of pretreating with Dako/Agilent Protein Block is performed for 5 minutes, 10 minutes, 15 minutes, 20 minutes or 30 minutes.

In some embodiments, the first detection agent is an antibody or antigen binding fragment thereof.

In some embodiments, the sample was contacted with the first detection agent at room temperature or at 37° C.

In some embodiments, the sample was contacted with the first detection agent for 15 minutes, 30 minutes, 45 minutes, 60 minutes, 95 minutes, or 120 minutes.

In some embodiments, the second detection agent is an antibody or antigen binding fragment thereof.

In some embodiments, the sample bound to the first detection agent was contacted with the second detection agent for 5 minutes, 10 minutes, 15 minutes, 20 minutes, 25 minutes, or 30 minutes.

In some embodiments, the sample comprises cells from a bodily fluid or tissue. In some embodiments, the tissue is brain tissue. In other embodiments, the tissue is from striatum, thalamus, midbrain, or medulla regions of the brain.

In some embodiments, the tissue is tumor tissue.

In some embodiments, the sample is from a subject. In some embodiments, the subject is a mammal. In some embodiments, the mammal is a human. In some embodiments, the mammal is Macaca fascicularis.

In one aspect, provided herein is a kit for performing the method disclosed herein.

In one aspect, provided herein is a method of treating a subject with a BCMA targeting agent, comprising a step of detecting BCMA in a sample from the subject.

In some embodiments, the step of detecting BCMA in the sample comprises the method disclosed herein.

In some embodiments, the targeting agent is a chimeric antigen receptor (CAR) T-cell. In other embodiments, the targeting agent is a T cell redirecting antibody.

In some embodiments, the subject has cancer. In some embodiments, the cancer is multiple myeloma.

### BRIEF DESCRIPTION OF THE DRAWINGS

The foregoing summary, as well as the following detailed description of specific embodiments of the present application, will be better understood when read in conjunction with the appended drawings. It should be understood, however, that the application is not limited to the precise embodiments shown in the drawings.

FIGS. 1A-1R show detection of B cell maturation antigen (“BCMA”) protein by immunohistochemistry (“IHC”) and BCMA RNA by in situ hybridization (“ISH”) on formalin-fixed, paraffin-embedded (FFPE) cell pellets and tissue controls. FIG. 1A shows BCMA expression in H929 cells. FIG. 1B shows BCMA expression in MM1R cells.

FIG. 1C shows BCMA expression in Jeko-1 cells. Dark chevrons indicate BCMA detection in the perinuclear region. FIG. 1D shows BCMA expression in Raji cells. Dark chevrons indicate BCMA detection in the perinuclear region. FIG. 1E shows BCMA expression in K562 cells. FIG. 1F shows BCMA expression in U-937 cells. FIG. 1G shows BCMA expression in HEK293 cells. FIGS. 1L-FIG. 1R show corroborative ISH on H929, MMIR, Jeko-1, Raji, K562, U-937, and HEK293 cells, respectively. Light chevrons indicate detection of BCMA RNA in FIG. 1N and FIG. 1O. FIG. 1H shows BCMA IHC on human colon FFPE samples. BCMA expression is membranous and perinuclear in putative resident plasma cells within the lamina propria. The inset of FIG. 1H shows higher magnification of the putative plasma cells. FIG. 1I shows ISH of BCMA expression on human colon FFPE, with positive putative plasma cells. The inset of FIG. 1I shows higher magnification of the putative plasma cells. FIG. 1J and FIG. 1K show IHC of BCMA expression on BCMA-negative cells transfected with BAFFR (FIG. 1J) or TACI (FIG. 1K). The insets of FIG. 1J and FIG. 1K show no immunoreactivity is detected, despite successful transfection of the cell lines as indicated by anti-Tag immunoreactivity (insets).

FIGS. 2A-2I show results of an IHC assay performed with various BCMA antibodies on brain samples. FIG. 2A shows BCMA immunoreactivity with Cell Signaling E6D7B clone (tags) in putamen. FIGS. 2B-2C show BCMA immunoreactivity with Cell Signaling E6D7B clone (tags) in medulla. FIG. 2D and FIG. 2G show BCMA immunoreactivity with Santa Cruz Biotech clone D6 (tags) in putamen. FIGS. 2E-2F and

FIGS. 2H-2I show BCMA immunoreactivity with Santa Cruz Biotech clone D6 (tags) in medulla.

FIGS. 3A-3H show colocalization of BCMA with trans- and cis-golgi markers.

FIGS. 3A-3D show results of immunofluorescence staining in H929 cells with various antibodies. FIG. 3A shows DAPI staining. FIG. 3B shows GOLM1 staining. FIG. 3C shows E6D7B staining. FIG. 3D shows co-localization of GOLM1 and E6D7B. The arrows indicate colocalization of GOLM1 and E6D7B. FIGS. 3E-3H show results of immunofluorescence staining in medulla with various antibodies. FIG. 3E shows DAPI staining. FIG. 3F shows GOLM1 staining. FIG. 3G shows E6D7B staining. FIG. 3H shows co-localization of GOLM1, E6D7B and BCMA. Chevrons indicate autofluorescence in scattered neurons.

FIGS. 4A-4C show BCMA RNA expression in various brain regions. FIG. 4A shows results of RNA sequencing in GTEx, Allen BrainSpan, and aggregated striatum data for all brain regions. FIG. 4B shows Allen BrainSpan bulk RNA-seq data for all brain regions plotted by development stage. FIG. 4C shows aggregated BCMA RNA expression data for striatum from Allen BrainSpan and components of the striatum (GTEx: caudate nucleus and putamen) plotted by donor age. Abbreviations: TPM, transcripts per million.

FIGS. 5A-5P show assay validation for the Santa Cruz D6 clone. FIG. 5A shows BCMA-expressing multiple myeloma cells in bone marrow using the D6 clone. FIG. 5B shows BCMA-expressing putative plasma cells in the lamina propria of the colon using the D6 clone. FIGS. 5C-51 and FIGS. 5J-5P show BCMA expression in cell pellets using the D6 clone.

FIGS. 6A-6F show comparison of E6D7B immunoreactivity with markers for protein aggregation. FIGS. 6A-6C show BCMA immunoreactivity observed in neurons of human brain using the E6D7B clone. FIG. 6D shows phosphorylated tau protein (pTau) immunoreactivity (chevrons) in the medulla. FIGS. 6E-6F show Bielschowsky silver stain in the medulla.

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010). Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The following references are incorporated herein by reference in their entirety: Bingham et al., Oncotarget 2017; 8 (55): 93392-403; Bolon et al., Toxicol Pathol. 2013; 41 (7): 1028-48; Gras et al., 1995; 7 (7): 1093-106; Uchihara et al., Neuropathology. 2014; 34 (6): 571-7; Schuh et al., J Immunol. 2017; 198 (8): 3081-8; and Shah et al., Leukemia. 2020; 34 (4): 985-1005.

### Definitions

It is noted here that as used in this specification and the appended claims, the singular forms “a,” “an,” and “the” include plural reference unless the context clearly dictates otherwise.

In the event there is a plurality of definitions for a term herein, those in this section prevail unless stated otherwise.

The term “about” or “approximately” means an acceptable error for a particular value as determined by one of ordinary skill in the art, which depends in part on how the value is measured or determined. In certain aspects, the term “about” or “approximately” means within 1, 2, 3, or 4 standard deviations. In certain aspects, the term “about” or “approximately” means within 50%, 20%, 15%, 10%, 9%, 8%, 7%, 6%, 5%, 4%, 3%, 2%, 1%, 0.5%, or 0.05% of a given value or range.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “administer” or “administration” refers to the act of injecting or otherwise physically delivering a substance as it exists outside the body into a patient, such as by oral, mucosal, intradermal, intravenous, intramuscular delivery and/or any other method of physical delivery described herein or known in the art. When a disease, or a symptom thereof, is being treated, administration of the substance typically occurs after the onset of the disease or symptoms thereof. When a disease, or a symptom thereof, is being prevented, administration of the substance typically occurs before the onset of the disease or symptoms thereof.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed antibody can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, single domain antibodies (e.g., VHH) and fragments thereof (e.g., domain antibodies). An antibody can be human, humanized, chimeric and/or affinity matured, as well as an antibody from other species, for example, mouse, rabbit, llama, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Pluckthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

As used herein, the term “BCMA” refers to B-cell maturation antigen, also known as tumor necrosis factor receptor superfamily member 17 (TNFRSF17), is a protein that in humans is encoded by the TNFRSF17 gene. BCMA is a cell surface receptor of the TNF receptor superfamily which recognizes B-cell activating factor. BCMA is preferentially expressed in mature B lymphocytes. The term “BCMA” includes any BCMA variant, isoform, and species homolog, which is naturally expressed by cells (including B cells) or can be expressed on cells transfected with genes or cDNA encoding the polypeptide. Unless noted, preferably the BCMA is a human BCMA. An exemplary human BCMA nucleotide sequence is provided by GenBank Accession Number BC058291. There are four major haplotypes of the BCMA gene in the human genome, and in the present disclosure the term “BCMA” is meant to encompass all four (Kawasaki et al., Genes Immun. 2:276-9, 2001).

“Antigen binding domain” or “antigen binding fragment” or “domain that binds an antigen” refers to a portion of a molecule that specifically binds an antigen. Antigen binding domain may include portions of an immunoglobulin that bind an antigen, such as a VH, a VL, the VH and the VL, Fab, Fab′, F(ab′)2, Fd and Fv fragments, domain antibodies (dAb) consisting of one VH or one VL, shark variable IgNAR domains, camelized VH domains, VHH, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3 and non-antibody scaffolds that bind an antigen.

As used herein, an “epitope” is a term in the art and refers to a localized region of an antigen to which a binding molecule (e.g., an antibody comprising a single chain antibody sequence) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Single domain antibody” or “sdAb” as used herein refers to a single monomeric variable antibody domain and which is capable of antigen binding. Single domain antibodies include VHH domains as described herein. Examples of single domain antibodies include, but are not limited to, antibodies naturally devoid of light chains such as those from Camelidae species (e.g., llama), single domain antibodies derived from conventional 4-chain antibodies, engineered antibodies and single domain scaffolds other than those derived from antibodies. Single domain antibodies may be derived from any species including, but not limited to mouse, human, camel, llama, goat, rabbit, and bovine. For example, a single domain antibody can be derived from antibodies raised in Camelidae species, for example in camel, llama, dromedary, alpaca and guanaco, as described herein. Other species besides Camelidae may produce heavy chain antibodies naturally devoid of light chain; VHHs derived from such other species are within the scope of the disclosure. In some embodiments, the single domain antibody (e.g., VHH) provided herein has a structure of FR1-CDR1-FR2-CDR2-FR3-CDR3-FR4. Single domain antibodies may be genetically fused or chemically conjugated to another molecule (e.g., an agent) as described herein. Single domain antibodies may be part of a bigger binding molecule (e.g., a multispecific antibody or a functional exogenous receptor).

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (koff) to association rate (kon) of a binding molecule (e.g., an antibody) to a monovalent antigen (koff/kon) is the dissociation constant KD, which is inversely related to affinity. The lower the KD value, the higher the affinity of the antibody. The value of KD varies for different complexes of antibody and antigen and depends on both kon and koff. The dissociation constant KD for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

The term “body fluid” or “bodily fluid” as used herein refers to a fluid that is obtained from a patient, such as a mammal (e.g., human) patient. For example, a body fluid may be blood, cerebral spinal fluid (CSF), breast milk or urine. The body fluid can also be blood fractionated to remove cells (i.e., plasma) or cells and clotting factors (i.e., serum).

The term “capture moiety” or “first antibody” as used herein refers to a composition that is capable of being specifically bound by another composition that is immobilized, e.g., attached or otherwise linked, to a solid support. Many of the detection moieties provided herein can also be used as capture moieties so long as a binding event is involved. For example, useful capture moieties include affinity labels for which specific and selective ligands are available (e.g., biotin with avidin, glutathione with GST), haptens and proteins for which antisera or monoclonal antibodies are available (e.g., c-Myc), nucleic acid molecules with a sequence complementary to a target, and peptides for which specific and selective ligands are available (e.g., histidine tag with Ni). Molecules that affect the binding characteristics to a chromatographic resin are also envisioned. The solid support can be, for example, a filter, a plate, a membrane, a chromatographic resin, or a bead.

The term “cutpoint factor” or “threshold” as used herein generally refers to a value that is used to mathematically manipulate the signal from the naïve pooled matrix (e.g., serum or plasma) to set the minimum signal required from a sample to be considered positive.

The term “derivative” when used in connection with antibody substances and polypeptides used in the methods provided herein refers to polypeptides chemically modified by techniques including, but not limited to, ubiquitination, conjugation to therapeutic or diagnostic agents, labeling (e.g., with radionuclides or various enzymes), covalent polymer attachment such as pegylation (i.e., derivatization with polyethylene glycol) and insertion or substitution by chemical synthesis of amino acids such as ornithine, which do not normally occur in human proteins. Derivatives can retain the binding properties of underivatized molecules.

The terms “detectable moiety,” “detection moiety” or a “label” as used herein refers to a composition (e.g., polypeptide or antibody) detectable by means including, but not limited to, spectroscopic, photochemical, biochemical, immunochemical, chemical, or other physical means. For example, useful detectable moieties or labels include Ruthenium (Ru)-based catalyst, Europium, ³²P, ³⁵S, fluorescent dyes, electron-dense reagents, enzymes (e.g., as commonly used in an ELISA), biotin-Streptavidin, digoxigenin, haptens and proteins for which antisera or monoclonal antibodies are available, and nucleic acid molecules with a sequence complementary to a target. The detectable moiety or label often generates a measurable signal, such as a radioactive, chromogenic, luminescent, or fluorescent signal, which can be used to quantitate the amount of bound detectable moiety or label in a sample.

Examples of labels which may be used in the invention include fluorophores, chromophores, electrochemiluminescent labels, bioluminescent labels, polymers, polymer particles, bead or other solid surfaces, gold or other metal particles or heavy atoms, spin labels, radioisotopes, enzyme substrates, haptens, antigens, Quantum Dots, aminohexyl, pyrene, nucleic acids or nucleic acid analogs, or proteins, such as receptors, peptide ligands or substrates, enzymes, and antibodies (including antibody fragments).

Some labels according to this invention comprise “color labels,” in which the target is detected by the presence of a color, or a change in color in the sample. Examples of “color labels” are chromophores, fluorophores, chemiluminescent compounds, electrochemiluminescent labels, bioluminescent labels, and enzymes that catalyze a color change in a substrate. In some embodiments, more than one type of color may be used, for instance, by attaching distinguishable color labels to a single detection unit or by using more than one detection unit, each carrying a different and distinguishable color label.

“Fluorophores” as described herein are molecules that emit detectable electro-magnetic radiation upon excitation with electro-magnetic radiation at one or more wavelengths. A large variety of fluorophores are known in the art and are developed by chemists for use as labels and can be conjugated to the linkers of the present invention. Examples include fluorescein or its derivatives, such as fluorescein-5-isothiocyanate (FITC), 5-(and 6)-carboxyfluorescein, 5- or 6-carboxyfluorescein, 6-(fiuorescein)-5-(and 6)-carboxamido hexanoic acid, fluorescein isothlocyanate, rhodamine or its derivatives such as tetramethylrhodamine and tetramethylrhodamine-5-(and-6)-isothiocyanate (TRITC). Other example fluorophores that could be conjugated to the instant linkers include: coumarin dyes such as (diethyl-amino) coumarin or 7-amino-4-methylcoumarin-3-acetic acid, succinimidyl ester (AMCA); sulforhodamine 101 sulfonyl chloride (TexasRed™ or TexasRed™ sulfonyl chloride; 5-(and-6)-carboxyrhodamine 101, succinimidyl ester, also known as 5-(and-6)-carboxy-X-rhodamine, succinimidyl ester (CXR); lissamine or lissamine derivatives such as lissamine rhodamine B sulfonyl Chloride (LisR); 5-(and-6)-carboxyfluorescein, succinimidyl ester (CFI); fluorescein-5-isothiocyanate (FITC); 7-diethylaminocoumarin-3-carboxylic acid, succinimidyl ester (DECCA); 5-(and-6)-carboxytetramethylrhodamine, succinimidyl ester (CTMR); 7-hydroxycoumarin-3-carboxylic acid, succinimidyl ester (HCCA); 6->fluorescein-5-(and-6)-carboxamidolhexanoic acid (FCHA); N-(4,4-difluoro-5,7-dimethyl-4-bora-3a,4a-diaza-3-indacenepropionic acid, succinimidyl ester; also known as 5,7-dimethylBODIPY™ propionic acid, succinimidyl ester (DMBP); “activated fluorescein derivative” (FAP), available from Molecular Probes, Inc.; eosin-5-isothiocyanate (EITC); erythrosin-5-Isothiocyanate (ErITC); and Cascade™ Blue acetylazide (CBAA) (the O-acetylazide derivative of 1-hydroxy-3,6,8-pyrenetrisulfonic acid). Yet other potential fluorophores useful in this invention include fluorescent proteins such as green fluorescent protein and its analogs or derivatives, fluorescent amino acids such as tyrosine and tryptophan and their analogs, fluorescent nucleosides, and other fluorescent molecules such as Cy2, Cy3, Cy 3.5, Cy5, Cy5.5, Cy 7, IR dyes, Dyomics dyes, phyeoerythrine, Oregon green 488, pacific blue, rhodamine green, and Alexa dyes. Yet other examples of fluorescent labels which may be used in the invention include and conjugates of R-phycoerythrin or aliiophycoerythrin, inorganic fluorescent labels such as particles based on semiconductor material like coated CdSe nanocrystallites.

A number of the fluorophores above, as well as others, are available commercially, from companies such as Molecular Probes, Inc. (Eugene, Oreg.), Pierce Chemical Co. (Rockford, Ill.), or Sigma-Aldrich Co. (St. Louis, Mo.).

Examples of polymer particles labels which may be used in the invention include micro particles, beads, or latex particles of polystyrene, PMMA or silica, which can be embedded with fluorescent dyes, or polymer micelles or capsules which contain dyes, enzymes or substrates.

Examples of metal particles which may be used in the invention include gold particles and coated gold particles, which can be converted by silver stains.

Examples of haptens that may be conjugated in some embodiments are fluorophores, myc, nitrotyrosine, biotin, avidin, strepavidin, 2,4-dinitrophenyl, digoxigenin, bromodeoxy uridine, sulfonate, acetylaminoflurene, mercury trintrophonol, and estradiol.

Examples of enzymes which may be used in the Invention comprise horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase and glucose oxidase (GO).

Examples of commonly used substrates for horse radish peroxidase (HRP) include 3,3′-diaminobenzidine (DAB), diaminobenzidine with nickel enhancement, 3-amino-9-ethylcarbazole (AEC), Benzidine dihydrochloride (BDHC), Hanker-Yates reagent (HYR), Indophane blue (IB), tetramethylbenzidine (TMB), 4-chloro-1-naphtol (CN), α-naphtol pyronin (α-NP), o-dianisidine (OD), 5-bromo-4-chloro-3-indolylphosphate (BCIP), Nitro blue tetrazolium (NBT), 2-(p-iodophenyl)-3-p-nitrophenyl-5-phenyl tetrazolium chloride (INT), tetranitro blue tetrazolium (TNBT), 5-bromo-4-chloro-3-indoxyl-beta-D-galactoside/ferro-ferricyanide (BCIG/FF).

Examples of commonly used substrates for Alkaline Phosphatase include Naphthol-AS-B1-phosphate/fast red TR(NABP/FR), Naphthol-AS-MX-phosphate/fast red TR(NAMP/FR), Naphthol-AS-B1-phosphate/fast red TR(NABP/FR), Naphthol-AS-MX-phosphate/fast red TR(NAMP/FR), Naphthol-AS-B1-phosphate/new fuschin (NABP/NF), bromochloroindolyl phosphate/nitroblue tetrazolium (BCIP/NBT), 5-Bromo-4-chloro-3-indolyl-b(beta)-d(delta)-galactopyranoside (BCIG).

Examples of luminescent labels which may be used in the invention include luminol, isoluminol, acridinlum esters, 1,2-dioxetanes and pyridopyridazines. Examples of electrochemiluminescent labels include ruthenium derivatives.

Examples of radioactive labels which may be used in the invention include radioactive isotopes of iodide, cobalt, selenium, hydrogen, carbon, sulfur and phosphorous.

The term “detectable antibody” as used herein refers to any antibody that can be detected. In some embodiments, the antibody is directly labeled with a detectable moiety. In certain embodiments, the antibody is a detectable anti-Ig antibody. The term “detectable anti-Ig antibody” as used herein refers to an anti-Ig antibody that can be detected. In some embodiments, the anti-Ig antibody is directly labeled with a detectable moiety in addition to its inherent binding to an Ig molecule. The Ig antibody can be of, for example, the IgG, IgE, IgM, IgD, IgA or IgY isotype.

As used herein, the term “primary antibody” refers to an antibody that binds directly to the antigen of interest. As used herein, the term “secondary antibody” refers to an antibody that is conjugated to a detection label. In some embodiments, the secondary antibody provided herein binds directly to the primary antibody. In other embodiments, the secondary antibody provided herein binds indirectly to the primary antibody, e.g., by binding to another antibody that recognizes the primary antibody.

In the context of a peptide or polypeptide, the term “fragment” as used herein refers to a peptide or polypeptide that comprises less than the full length amino acid sequence. Such a fragment may arise, for example, from a truncation at the amino terminus, a truncation at the carboxy terminus, and/or an internal deletion of a residue(s) from the amino acid sequence. Fragments may, for example, result from alternative RNA splicing or from in vivo protease activity. In certain embodiments, fragments include polypeptides comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of an antibody that immunospecifically binds to a target antigen. In a specific embodiment, the antibody fragment that immunospecifically binds to a target antigen, retains at least 1, at least 2, or at least 3 functions of the antibody.

The terms “identical” or percent “identity,” in the context of two or more polynucleotide or polypeptide sequences, refer to two or more sequences or subsequences that are the same or have a specified percentage of nucleotides or amino acid residues that are the same, when compared and aligned for maximum correspondence, as measured using a sequence comparison algorithms or by visual inspection.

The term “antibody that immunospecifically binds” with a target antigen and analogous terms are used interchangeably herein and refer to antibodies and fragments thereof, that specifically bind to only the target antigen or epitope. In yet other embodiments, antibodies provided herein immunospecifically bind to an Ig, such as an IgG, IgE, IgM, IgD, IgA isotype.

The term “interference” as used herein generally refers to the presence of substances in body fluid (e.g., serum or plasma) samples that prevent the target analyte from accurate detection and measurement. As used herein, interference generally refers to the effect of free drug or the effect of the matrix (e.g., serum or plasma) on the concentration-response relationship. For example, interference from matrix may be evaluated as the relative accuracy to samples without the potential interference to target a range of 75-125% relative accuracy.

The term “in vivo,” in the context of samples, refers to samples obtained from a subject, e.g., a patient, such as a human patient, including biological samples such as biological or body fluids, e.g., blood, plasma, serum, bone marrow, spinal fluid, brain fluid, or tissues, such as lymph tissue, a thin layer cytological sample, a fresh frozen tissue sample or a tumor tissue. The term “in vivo” is to be distinguished from the term “in vitro,” which encompasses cells or cell lines or biomolecular components of cells that have been cultured or propagated outside of a living organism.

The term “limit of detection,” “LOD” or “sensitivity” as used herein generally refers to the lowest analyte concentration in a body fluid (e.g., serum or plasma) sample that can be detected but not necessarily quantitated as an exact value. For example, LOD may be defined as the analyte concentration that consistently generates a signal greater than the measured mean response of the pooled naïve matrix plus a cutpoint factor.

The term “matrix” or “matrices” as used herein generally refers to the biological background in which the antibodies are measured. Examples of matrices include, for example, body fluid and tissue.

The term “monoclonal antibody” refers to an antibody obtained from a population of homogenous or substantially homogeneous antibodies, and each monoclonal antibody will typically recognize a single epitope on the antigen. In certain embodiments, a “monoclonal antibody,” as used herein, is an antibody produced by a single hybridoma or other cell. The term “monoclonal” is not limited to any particular method for making the antibody. For example, monoclonal antibodies used in the methods provided herein may be made by the hybridoma method as described in Kohler et al.; Nature, 256:495 (1975) or may be isolated from phage libraries using the techniques known in the art. Other methods for the preparation of clonal cell lines and of monoclonal antibodies expressed thereby are well known in the art (see, for example, Chapter 11 in: Short Protocols in Molecular Biology, (2002) 5th Ed., Ausubel et al., eds., John Wiley and Sons, New York).

“Polyclonal antibodies” as used herein refers to an antibody population generated in an immunogenic response to a protein having many epitopes and thus includes a variety of different antibodies directed to the same and to different epitopes within the protein. Methods for producing polyclonal antibodies are known in the art (See, e.g., see, for example, Chapter 11 in: Short Protocols in Molecular Biology, (2002) 5th Ed., Ausubel et al., eds., John Wiley and Sons, New York).

The term “precision” as used herein generally refers to the variability in signal between the analysts and days. For example, precision may be evaluated as coefficient of variation, ranges of values, or using ANOVA statistics.

As used herein, the terms “prevent,” “preventing,” and “prevention” refer to the total or partial inhibition of the development, recurrence, onset or spread of a disease and/or symptom related thereto (e.g., a disease or symptom related thereto that is associated with elevated phenylalanine levels, such as PKU or cancer, in a patient), resulting from the administration of a therapy or combination of therapies provided herein.

The term “reagent stability” as used herein generally refers to the robustness of preparation and storage stability of the reagents. For example, reagent stability may be established by the conditions that still permit values to be measured within 75-125% accuracy relative to freshly prepared reagents.

The term “robustness” as used herein generally refers to the capacity of the assay to remain unaffected by small variations in method parameters and indicates reliability of the assay during normal run conditions. For example, robustness can be evaluated as the percent change of reagent concentration, reagent volume, or incubation time that still generates signal within 75-125% accuracy relative to the nominal conditions.

The term “sample” as used herein generally refers to a test fluid or tissue, e.g., taken from a patient, that can be used in the methods provided herein. In some embodiments, the sample is an in vivo sample, for example, bodily (or biological) fluid from a subject, e.g., a patient, such as a human patient. Non-limiting examples of such bodily fluids include blood (e.g., human peripheral blood (HPB)), blood lysate, serum, blood plasma, fine needle aspirate, ductal lavage, spinal fluid, brain fluid, bone marrow, ascites fluid or any combination thereof. In other embodiments, the sample is taken from a biopsy tissue such as a tumor tissue from a subject or a thin layer cytological sample of other body tissue or organ. In certain embodiments, the sample comprises a peripheral blood sample, tumor tissue or suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, a paraffin embedded tissue sample. In other embodiments, the sample is an extract or processed sample produced from any of a peripheral blood sample, a tumor tissue or a suspected tumor tissue, a thin layer cytological sample, a fine needle aspirate sample, a bone marrow sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample or a paraffin embedded tissue sample.

The term “specificity” as used herein generally refers to the ability of the assay to detect antibodies that react with a specific protein. For example, specificity may refer to a proportional detection response with the specific analyte, while response to a non-specific protein should be below the LOD. The proportional response may be evaluated against a correlation coefficient R value greater than or equal to 0.98. When used in connection with the method provided herein to detect target antigen, specificity refers to the ability to detect antigens that react with a specific protein.

As used herein, the terms “subject” and “patient” are used interchangeably. As used herein, a subject is preferably a mammal such as a non-primate (e.g., cows, pigs, horses, cats, dogs, rats, etc.) or a primate (e.g., monkey and human), most preferably a human. In one embodiment, the subject is a mammal, preferably a human. In some embodiments of the methods and kits provided herein, the patient has a disease or symptom, or cancer. In other embodiments of the methods and kits provided herein, the patient is a patient undergoing cancer therapy. In yet other embodiments of the methods and kits provided herein, the patient is a pregnant female or an infant (e.g., age 0 to about 36 months).

As used herein, the term “tag” and “label” are used interchangeably and refer to any type of moiety that is attached to an antibody or antigen binding fragment thereof, or other polypeptide used in the methods provided herein. The term “detectable” or “detection” with reference to an antibody or tag refers to any antibody or tag that is capable of being visualized or wherein the presence of the antibody or tag is otherwise able to be determined and/or measured (e.g., by quantitation). Non-limiting examples of a detectable tag include fluorescent or other chemiluminescent tags, and tags that can be amplified and quantitated using PCR. In certain embodiments, the secondary antibody used in the methods provided herein is a biotinylated secondary antibody that is used in combination with a labeled streptavidin.

As used herein, the term “therapy” refers to any protocol, method and/or agent that can be used in the prevention, management, treatment and/or amelioration of disease (or symptom related thereto) or cancer. In certain embodiments, the terms “therapies” and “therapy” refer to a biological therapy, supportive therapy, and/or other therapies useful in the prevention, management, treatment and/or amelioration of a disease or cancer known to one of skill in the art such as medical personnel.

The term “tissue” as used herein refers to tissues that are obtained from a mammal, e.g., human. For example, a tissue may be from a biopsy sample, surgically removed tissue, or postmortem collection. Furthermore, the tissue may be homogenized and extracted to isolate the enzyme or antibodies from the tissue.

As used herein, the terms “treat,” “treatment” and “treating” refer to the reduction or amelioration of the progression, severity, and/or duration of a disease (or symptom related thereto) or cancer resulting from the administration of one or more therapies.

The term “variant” as used herein refers to a polypeptide sequence that contains at least one amino acid substitution, deletion, or insertion in the coding region relative to the original polypeptide coding domains. Variants retain the biological activity of the naturally occurring polypeptide.

As used herein, the term “in situ hybridization” or “ISH” refers to a technique for localizing and visualizing specific target nucleic acids with the preservation of morphology of the source samples.

As used herein, the term “immunohistochemistry” or “IHC” refers to a technique for detecting proteins of interest in source samples utilizing antibodies, with the preservation of morphology of the source samples. Immunofluorescence (IF) refers to fluorescent labeling, thus it is also encompassed in the term of IHC.

As used herein, the term “crosslink” refers to a process of binding two or more molecules together. The “crosslinking agent” or equivalent refers to agents containing two or more chemically reactive ends that attach themselves to the functional groups found in proteins and other molecules. Specifically, if the crosslinking agent is formaldehyde or its equivalent, a nucleophilic group on an amino acid or nucleic acid base forms a covalent bond with formaldehyde, which is stabilized in a second step that involves another functional group, often on another molecule, leading to formation of a methylene bridge. If the crosslinking agent is an oxidizing agent, it can react with the side chains of proteins and other biomolecules, allowing the formation of crosslinks that stabilize tissue structure.

As used herein, the term “fixation” or “fixing” when made in reference to fixing a sample in the IHC process refers to a procedure to preserve a sample from decay due to, e.g., autolysis or putrefaction. It terminates any ongoing biochemical reactions and may also increase the treated tissues' mechanical strength or stability.

The terms “detecting” as used herein generally refer to any form of measurement, and include determining whether an element is present or not. This term includes quantitative and/or qualitative determinations.

### IHC Methods for Detecting BCMA

Immunohistochemistry (IHC) on formalin fixed paraffin embedded (FFPE) tissue is critical step in R&D therapeutic campaigns by identify cells expressing the target proteins of interest and predicting potential toxicities. A robust IHC assay depends on suitable primary antibodies that reliably recognize the target with optimal specificity and sensitivity. FFPE tissues often present over-fixed proteins with altered conformation, which renders repurposing of antibodies validated in non-IHC assays extremely uncertain. During the tissue preparation and preservation process, if a crosslinking agent, such as formalin, is used, formalin fixation may mask epitopes and result in decreased immunoreactivity (see Arnold et al., Biotech Histochem 71:224-230 (1996)). Formalin fixation is a time-dependent process in which increased fixation time results in continued formaldehyde group binding to proteins to a point of equilibrium (see Fox et al., J Histochem Cytochem 33:845-853 (1985)). Studies have shown that formalin fixation, especially if prolonged, results in decreased antigenicity (see Battifora and Kopinski, J Histochem Cytochem 34:1095-1100 (1986)), which limits the use of formalin-fixed tissues for diagnostic IHC (see Ramos-Vara, Vet Pathol 42:405-426 (2005), Webster et al., J Histochem Cytochem. 57 (8): 753-761 (2009)). Generation of new IHC antibodies, when suitable reagents are not commercially available, requires screening many candidates against relevant controls.

In one aspect, provided herein is a method of detecting BCMA in a formalin-fixed paraffin-embedded (“FFPE”) sample. In some embodiments, the method comprises: (1) sectioning and mounting the FFPE sample, (2) deparaffinizing the sample, (3) heating the sample to mediate antigen retrieval, (4) pretreating the sample with a blocking agent, (5) contacting the sample with a first detection agent, (6) contacting the sample bound to the first detection agent with a second detection agent, and (7) detecting the presence of the unbound second detection agent bound to the sample.

In another embodiment, the method comprises: (1) sectioning and mounting the FFPE sample, (2) deparaffinizing the sample, (3) heating the sample to mediate antigen retrieval, (4) pretreating the sample with a blocking agent, (5) contacting the sample with a first detection agent, (6) removing unbound sample, (7) contacting the sample bound to the first detection agent with a second detection agent, (8) removing the unbound second detection agent and (9) detecting the presence of the unbound second detection agent bound to the sample.

In some embodiments, a BCMA detected by a method of this disclosure includes any BCMA variant, isoform, and species homolog, which is naturally expressed by cells (including B cells) or can be expressed on cells transfected with genes or cDNA encoding the polypeptide. Unless noted, preferably the BCMA is a human BCMA.

In some embodiments, a sample used in a method of this disclosure comprises cells collected from bodily fluid or tissue. In some embodiments, a sample may comprise any tissue found in an organism. In some embodiments, the organism comprises a vertebrate or non-vertebrate organism. In some embodiments, the vertebrate comprises a human or a non-human primate. In some embodiments, the monkey comprises a cynomolgus monkey (Macaca fascicularis), a rhesus monkey (Macaca mulatta), a marmoset (Callithrix jacchus), a Chimpanzee (Pan troglodytes), a Bonobo (Pan paniscus), a Bornean orangutan (Pongo pygmaeus), a sumatran orangutan (Pongo abelii), a Tapanuli orangutan (Pongo tapanuliensis) or any other known primate species. In some embodiments, the tissue comprises a tumor tissue. In some embodiments, the tumor tissue comprises a benign tumor. In another embodiment, the tumor tissue comprises a premalignant tumor. In other embodiments, the tumor tissue comprises a malignant tumor. A nonexhaustive list of exemplary tumor tissues contemplated by the present disclosure includes a sarcoma, carcinoma, adenocarcinoma, lymphoma, breast tumor, mammary tumor, prostate tumor, head and neck tumor, brain tumor, pituitary tumor, glioblastoma, medulloblastoma, atypical teratoid/rhabdoid tumor, bladder tumor, pancreatic tumor, islet tumor, liver tumor, ovarian tumor, colorectal tumor, lung tumor, bronchial tumor, tracheobronchial tumor, skin tumor, lymphoid tumor, and a gastrointestinal tumor.

In further embodiments, the tissue may comprise brain tissue. The brain tissue may further comprise the striatum region, thalamus region, midbrain region or medulla region of the brain.

In some embodiments, the sample used in a method of this disclosure is obtained from a subject. In some embodiments, the subject is a mammal. In some embodiments, the mammal is a human or a non-primate human. In other embodiments, the mammal is Macaca fascicularis. Samples may be collected by any method known in the art. A person skilled in the art will appreciate that collection procedures will vary according to the sample type and the intended analysis. For example, blood samples may be collected by intravenous route with an evacuated tube system. Brain tissue may be collected by making an incision in the scalp, drilling a hole in the skull and inserting a needle into the brain to obtain brain tissue.

In some embodiments of this disclosure, tissue or cell samples may be fixed or embedded. Fixatives may be needed, for example, to preserve cells and tissues in a reproducible and life-like manner. Fixatives may also stabilize cells and tissues, thereby protecting them from the rigors of processing and staining techniques. For example, samples comprising tissue blocks, sections, or smears may be immersed in a fixative fluid, or in the case of smears, dried.

Many methods of fixing and embedding tissue specimens are known, for example, formalin-fixation and subsequent paraffin embedding (FFPE). Any suitable fixing agent may be used. Examples include ethanol, acetic acid, picric acid, 2-propanol, 3,3′-diaminobenzidine tetrahydrochloride dihydrate, acetoin (mixture of monomer) and dimer, acrolein, crotonaldehyde (cis+trans), formaldehyde, glutaraldehyde, glyoxal, potassium dichromate, potassium permanganate, osmium tetroxide, paraformaldehyde, mercuric chloride, tolylene-2,4-diisocyanate, trichloroacetic acid, tungstic acid. Other examples include formalin (aqueous formaldehyde) and neutral buffered formalin, glutaraldehyde, carbodiimide, imidates, benzoequinone, osmic acid and osmium tetraoxide. Fresh biopsy specimens, cytological preparations (including touch preparations and blood smears), frozen sections, and tissues for IHC analysis may be fixed in organic solvents, including ethanol, acetic acid, methanol and/or acetone sample

In some embodiments of this disclosure, the method comprises a pre-treating step to increase the reactivity or accessibility of target molecules in the sample, while reducing nonspecific interactions. This process is referred to as antigen retrieval, which is also known as target retrieval, epitope retrieval, target unmasking or antigen unmasking. See, e.g., Shi et al., J Histochem Cytochem, 45 (3): 327 (1997). Antigen retrieval encompasses a variety of methods including enzymatic digestion with proteolytic enzymes, such as e.g. proteinase, pronase, pepsin, papain, trypsin or neuraminidase. Some embodiments may use heat, e.g. “heat-mediated antigen retrieval”. Heating may involve a microwave irradiation, or a water bath, a steamer, a regular oven, an autoclave, or a pressure cooker in an appropriately pH stabilized buffer. In some embodiments, the heat-mediated antigen retrieval is performed using an EDTA-based solution.

In some embodiments, the EDTA-based solution has a pH between 8.0 to 9.0, 8.5 to 9.5, 9.0 to 10.0, 9.5 to 10.5, 10 to 11, 10.5 to 11.5, 12.0 to 13.0, 12.5 to 13.5 or 13.0 to 14.0. In another embodiment, the EDTA-based solution has a pH of 8.0, 8.5, 9.0, 9.5, 10.0, 10.5, 11.0, 11.5, 12.0, 12.5, 13.0, 13.5 or 14.0. Additional pH stabilized buffers may include EGTA, Tris-HCl, citrate, urea, glycine-HCl or boric acid.

In some embodiments, the step of heat-mediated antigen retrieval is performed for 1 minute, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes, or 60 minutes. In other embodiments, the heat-mediated antigen retrieval step is performed at a temperature between 80° C. and 120° C. In some embodiments, the heat-mediated antigen retrieval step is performed at a temperature between 80° C. and 95° C., 85° C. and 100° C., 90° C. and 105° C., 95° C. and 110° C., 100° C. and 115° C., or 105° C. and 120° C. In other embodiments, the heat-mediated antigen retrieval method is performed at 98° C., 99° C., 100° C., or 120° C. In a specific embodiment, the heat-mediated antigen retrieval method is performed at 100° C. In some embodiments, any combination of the above-described antigen retrieval methods may be used.

In some embodiments of this disclosure, the method comprises blocking a sample with a blocking agent. Blocking agents known in the art include serum (bovine serum albumin), casein, gelatin, salmon sperm DNA, avidin/biotin, among others. In some embodiments, the blocking agent blocks endogenous peroxidase. Thus, the blocking agent may comprise a peroxide block. Blocking may be performed for a sustained period of time. In some embodiments, blocking with a peroxide block may be performed for 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, or 20 minutes. In other embodiments, the blocking agent blocks non-specific antibody binding. Thus, the blocking agent may comprise a Dako/Agilent Protein Block. In some embodiments, blocking with the Dako/Agilent Protein block may be performed for 5 minutes, 10 minutes, 15 minutes, 20 minutes, 25 minutes, 30 minutes, 35 minutes, 40 minutes, 45 minutes, 50 minutes, 55 minutes, 60 minutes, 90 minutes, or 120 minutes. In some embodiments, blocking with the Dako/Agilent Protein block may be performed for 2 hours, 3 hours, 4 hours, 5 hours, 6 hours, 7 hours, 8 hours, 9 hours, 10 hours, 11 hours or 12 hours. It will be understood that additional blocking agents may be used. A person skilled in the art will appreciate that the blocking agent and time for blocking depend on the tissue being processed.

The present disclosure is compatible with many known detection formats and their associated samples. For example, the invention may be used in connection with immunoassays, protein detection assays, or nucleic acid hybridization assays such as: immunohistochemistry (IHC), immunocytochemistry (ICC), and in situ hybridization (ISH), labeling on surfaces or arrays, among others. All of those detection assays are useful in research as well as in the detection and diagnosis of a variety of diseases and conditions, for example.

For example, IHC specifically provides a method of detecting targets in a sample or tissue specimen in situ (see Mokry 1996, ACTA MEDICA 39:129). The overall cellular integrity of the sample is maintained in IHC, thus allowing detection of both the presence and location of the targets of interest. Typically a sample is fixed with formalin, embedded in paraffin and cut into sections for staining and subsequent inspection by light microscopy. Current methods of IHC use either direct labeling or secondary antibody-based or hapten-based labeling. Examples of known IHC systems include, for example, EnVision™ (DakoCytomation), Powervision® (Immunovision, Springdale, Ariz.), the NBA™ kit (Zymed Laboratories Inc., South San Francisco, Calif.), HistoFine® (Nichirei Corp, Tokyo, Japan). The present invention may allow for enhancement of signal or increased flexibility in IHC detection platforms.

IHC, ISH and etiological techniques may be performed in a matrix of tissue, cell and proteins which may be partly cross-linked and very inhomogeneous in nature. Diffusion rates increase with increasing concentrations and increasing temperature, but decrease with molecular weight and molecular size. Therefore, the physical size of the components is of great importance. For instance, large molecules can be excluded from diffusing into parts of the sample whereas small sized components more easily may diffuse in and out of the different compartments of the sample. In some embodiments, the units of the invention may be designed to be of small size and, for example, smaller than an antibody or biotin-streptavidine complex, in order to improve target recognition and detection.

In some embodiments of this disclosure, the method comprises contacting the sample with a detection agent. As used herein, the term “detection agent” refers to a binding agent capable of specific binding to a target. Such binding agents include, for example, antibodies and ligands. Antibodies include full length antibodies as well as functional fragments such as those exemplified above. Ligands include full length polypeptides such as those exemplified above and functional binding fragments thereof. Ligands also include the non-polypeptide ligands exemplified above. When referring to specific binding to a target, a detection agent of the disclosure can bind the target directly or it can be made specific to the target by indirect means. In some embodiments the sample is contacted with a first detection agent and a second detection agent. In some embodiments, detection agent comprises an antibody or an antigen binding fragment thereof. In some embodiments, the sample is contacted with a first detection agent at room temperature. In some embodiments, the sample is contacted with a first detection agent at 4° C. or 37° C. In some embodiments, the sample was contacted with the first detection agent for 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 90 minutes, 95 minutes, 100 minutes, 105 minutes, 110 minutes, 115 minutes or 120 minutes. In some embodiments, the sample was contacted with the first detection agent for 3 hours, 4 hours, 5 hours, 6 hours, 7 hours, 8 hours, 9 hours, 10 hours, 11 hours or 12 hours. In some embodiments, the sample is contacted with a second detection agent at room temperature. In some embodiments, the sample is contacted with a second detection agent at 37° C. In other embodiments, the sample is contacted with a second detection agent at 4° C. In some embodiments, the sample was contacted with the second detection agent for 1 minute, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, or 60 minutes.

In some embodiments, detection above background of an amount of the second detection agent bound to the sample indicates the presence of at least one molecule of BCMA in the sample.

In some embodiments, the method of detecting BCMA″) in a FFPE sample, comprises: sectioning and mounting of the FFPE samples of colon, spleen, and lymph node from human and cynomolgus monkey; deparaffinizing the samples; heating the sample at 100° C. for 20 minutes in an EDTA-based solution that has a pH of 9.0; pretreating the samples with endogenous peroxidase solution for 10 minutes, and then pretreating the samples with Dako serum-free protein block for 10 minutes; contacting the sample with a rabbit monoclonal anti-BCMA antibody for 30 minutes at a concentration of 7.8 μg/mL in antibody diluent; washing the sample extensively; contacting the sample bound to the first detection agent with a second detection agent comprising DAB.

### ISH

ISH involves contacting a sample containing a target nucleic acid sequence (e.g., genomic target nucleic acid sequence) in the context of a metaphase or interphase chromosome preparation (such as a cell or tissue sample mounted on a slide) with a probe (i.e., the target nucleic acid probe described above) that is specifically hybridizable or specific for the target nucleic acid sequence (e.g., genomic target nucleic acid sequence). The slides are optionally pretreated, e.g., to remove paraffin or other materials that can interfere with uniform hybridization. The chromosome sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess target nucleic acid probe, and detection of specific labeling of the chromosome target is performed. The probes and probe systems of the present disclosure can be used for nucleic acid detection, such as in situ hybridization procedures (e.g., fluorescence in situ hybridization (FISH), chromogenic in situ hybridization (CISH) and silver in situ hybridization (SISH)). Hybridization between complementary nucleic acid molecules is mediated via hydrogen bonding, which includes Watson-Crick, Hoogsteen or reversed Hoogsteen hydrogen bonding between complementary nucleotide units. For example, adenine and thymine are complementary nucleobases that pair through formation of hydrogen bonds. If a nucleotide unit at a certain position of a probe of the present disclosure is capable of hydrogen bonding with a nucleotide unit at the same position of a DNA or RNA molecule (e.g., a target nucleic acid sequence) then the oligonucleotides are complementary to each other at that position. The probe and the DNA or RNA are complementary to each other when a sufficient number of corresponding positions in each molecule are occupied by nucleotide units which can hydrogen bond with each other, and thus produce detectable binding. A probe need not be 100% complementary to its target nucleic acid sequence (e.g., genomic target nucleic acid sequence) to be specifically hybridizable. However sufficient complementarity is needed so that the probe binds, duplexes, or hybridizes only or substantially only to a target nucleic acid sequence when that sequence is present in a complex mixture (e.g., total cellular DNA or RNA).

In some embodiments of the present disclosure, detection is facilitated by hybridization of a detection agent to the target nucleic acid probe. The detection agent may be detected by direct detection or by indirect detection. For example, in some direct detection embodiments, the detection agent is labelled with one or more fluorescent compounds, and the sample is analyzed by fluorescence microscopy or imaging. In some indirect detection embodiments, the detection agent comprises a plurality of detectable moieties comprising first members of a binding pair (i.e., a hapten or biotin) which are detected by contacting the sample with a compound comprising a second member of the binding pair (i.e., anti-hapten antibody or avidin) conjugated to a detectable moiety (i.e., a fluorochrome or quantum dot). For a general description of in situ hybridization procedures, see, e.g., U.S. Pat. No. 4,888,278. Numerous procedures for FISH, CISH and SISH are known in the art. For example, procedures for performing FISH are described in U.S. Pat. Nos. 5,447,841, 5,472,842, 5,427,932, and for example, in Pinkel et al., Proc. Natl. Acad. Sci. 83:2934-2938, 1986; Pinkel et al., Proc. Natl. Acad. Sci. 85:9138-9142, 1988, and Lichter et al., Proc. Natl. Acad. Sci. 85:9664-9668, 1988. CISH is described in, e.g., Tanner et al., Am. J. Pathol. 157:1467-1472, 2000, and U.S. Pat. No. 6,942,970. Additional detection methods are provided in U.S. Pat. No. 6,280,929. Exemplary procedures for detecting viruses by in situ hybridization can be found in Poddighe et al., J. Clin. Pathol. 49:M340-M344, 1996.

Numerous reagents and detection schemes can be employed in conjunction with FISH, CISH, and SISH procedures to improve sensitivity, resolution, or other desirable properties. As discussed above, detection agents labeled with fluorophores (including fluorescent dyes and QUANTUM DOTS™) can be directly optically detected when performing FISH. Alternatively, the detection agent can be labeled with a non-fluorescent molecule, such as a hapten (such as the following non-limiting examples: biotin, digoxygenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Detection agents labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT™) or with another indirectly detectable moiety, or can be contacted with one or more additional specific binding agents (e.g., secondary or specific antibodies), which can in turn be labeled with a fluorophore. Optionally, the detectable label is attached directly to the antibody, receptor (or other specific binding agent). Alternatively, the detectable label is attached to the binding agent via a linker, such as a hydrazide thiol linker, a polyethylene glycol linker, or any other flexible attachment moiety with comparable reactivities. For example, a specific binding agent, such as an antibody, a receptor (or other anti-ligand), avidin, or the like can be covalently modified with a fluorophore (or other label) via a heterobifunctional polyalkylene glycol linker such as a heterobifunctional polyethylene glycol (PEG) linker. A heterobifunctional linker combines two different reactive groups selected, e.g., from a carbonyl-reactive group, an amine-reactive group, a thiol-reactive group and a photo-reactive group, the first of which attaches to the label and the second of which attaches to the specific binding agent.

In other examples, the detection agent, or specific binding agent (such as an antibody, e.g., a primary antibody, receptor or other binding agent) comprises an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and U.S. patent application publication number 2007011715.

It will be appreciated by those of skill in the art that by appropriately selecting labeled detection agents and/or labeled binding pairs, multiplex detection schemes can be produced to facilitate detection of multiple target nucleic acid sequences (e.g., genomic target nucleic acid sequences) in a single assay (e.g., on a single cell or tissue sample or on more than one cell or tissue sample). For example, a first detection agent that corresponds to a first target nucleic acid probe can be labeled with a first hapten, such as biotin, while a second detection agent that corresponds to a second target nucleic acid sequence can be labeled with a second hapten, such as DNP. Following exposure of the sample to the probe sets, the bound probes can be detected by contacting the sample with a first specific binding agent (in this case avidin labeled with a first fluorophore, for example, a first spectrally distinct QUANTUM DOT™, e.g., that emits at 585 nm) and a second specific binding agent (in this case an anti-DNP antibody, or antibody fragment, labeled with a second fluorophore (for example, a second spectrally distinct QUANTUM DOT″, e.g., that emits at 705 nm). Additional probes/binding agent pairs can be added to the multiplex detection scheme using other spectrally distinct fluorophores. Numerous variations of direct, and indirect (one step, two step or more) can be envisioned, all of which are suitable in the context of the disclosed probes and assays.

Standard fluorescence microscopes are an inexpensive tool for the detection of reagents and probes incorporating fluorescent compounds, such as quantum dot bioconjugates. Since quantum dot conjugates are virtually photo-stable, time can be taken with the microscope to find regions of interest and adequately focus on the samples. Quantum dot conjugates are useful any time bright photo-stable emission is required and are particularly useful in multicolor applications where only one excitation source/filter is available and minimal crosstalk among the colors is required.

### Methods of Treating

In one aspect, provided herein is a method of treating a subject with a BCMA targeting agent, comprising a step of detecting BCMA in a sample from the subject. In some embodiments, a method of treating a subject with a BCMA targeting agent first comprises detecting BCMA. Any of the methods of disclosed herein may be used in a method of detecting BCMA.

In some embodiments, the targeting agent comprises any targeting agent known in the art. As used herein, “targeting agent” refers to any molecule capable of interacting with a target of interest. Thus, in some embodiments, the targeting agent comprises a chimeric antigen receptor (CAR) T-cell or a T cell redirecting antibody. In other embodiments, the targeting agent comprises an antibody or an antigenic fragment thereof. The antibody or antigenic fragment thereof may comprise an anti-BCMA antibody or antigenic fragment thereof. In further embodiments, the antibody may comprise a bispecific or multispecific antibody. The bispecific or multispecific antibody may comprise an anti-BCMA bispecific or multispecific antibody. In other embodiments, the targeting agent may comprise a small molecule capable of binding BCMA. In other embodiments, the targeting agent may comprise a BCMA polypeptide.

In some embodiments, the target of interest is a soluble polypeptide. In other embodiments, the target of interest is a membrane-bound polypeptide including a receptor. In some embodiments, the receptor comprises BCMA.

In some embodiments, a subject of the present treatment method has a disease, disorder, condition or syndrome. In some embodiments, the disease, disorder, condition or syndrome may be any human disease, disorder, condition or syndrome known in the art. In some embodiments, the targeting agent of the present disclosure may also be used to treat diseases that express BCMA.

In some embodiments, the subject of the present treatment method has a cancer or tumor. In other embodiments, the cancer may comprise anal cancer, bile duct cancer, bladder cancer, bone cancer, breast cancer, cervical cancer, chronic lymphocytic leukemia (CLL), chronic myelogenous leukemia (CML), chronic myeloproliferative neoplasms, colon cancer, colorectal cancer, endometrial cancer, esophageal cancer, fallopian tube cancer, gallbladder cancer, gastric (stomach) cancer, head and neck cancer, liver cancer, hepatocellular carcinoma, Hodgkin lymphoma, laryngeal cancer, leukemia, lung cancer, non-small cell lung cancer, ovarian cancer, pancreatic cancer, melanoma, merkel cell carcinoma (skin cancer), mesothelioma, malignant, paranasal sinus and nasal cavity cancer (head and neck cancer), parathyroid cancer, penile cancer, pharyngeal cancer (head and neck cancer), prostate cancer, rectal cancer, renal cell cancer, skin cancer, small intestine cancer, stomach cancer, testicular cancer, thyroid cancer, urethral cancer, uterine cancer, vaginal cancer, and vulvar cancer.

In further embodiments, examples of breast cancers include but are not limited to invasive ductal carcinoma, invasive lobular carcinoma, ductal carcinoma in situ and lobular carcinoma in situ. The examples of respiratory tract cancers include but are not limited to small cell lung cancer, non-small cell lung cancer, bronchial adenoma and pleuropulmonary blastoma. The examples of brain cancers include but are not limited to brain stem and hypothalamic gliomas, cerebellar and cerebral astrocytomas, medulloblastoma, ependymoma and neuroectodermal and pineal tumors. Male genital neoplasms include but are not limited to prostatic cancers and testicular cancers. Female genital neoplasms include but are not limited to endometrial cancer, cervical cancer, ovarian cancer, vaginal cancer, vulvar cancer and hysteroma. Gastrointestinal tumors include but are not limited to anal cancer, colon cancer, colorectal cancer, esophageal cancer, gallbladder cancer, stomach cancer, pancreatic cancer, rectal cancer, small intestine cancer and salivary gland cancer. Urethral tumors include but are not limited to bladder cancer, penile cancer, renal carcinoma, renal pelvic carcinoma, ureteral cancer and urethral cancer. Eye cancers include but are not limited to intraocular melanoma and retinoblastoma. The examples of liver cancers include but are not limited to hepatocellular carcinoma (hepatocellular carcinoma with or without fibrolamellar variation), cholangiocarcinoma (intrahepatic cholangiocarcinoma) and combined hepatocellular-cholangiocarcinoma. Skin cancers include but are not limited to squamous-cell carcinoma, Kaposi's sarcoma, malignant melanoma, Merkel cell carcinoma and non-melanoma skin cancers. Head and neck cancers include but are not limited to laryngeal/hypopharyngeal/nasopharyngeal/oropharyngeal carcinomas, as well as lip and oral cancers. Lymphomas include but are not limited to AIDS-associated lymphoma, non-Hodgkin's lymphoma, cutaneous T-cell lymphoma, Hodgkin's disease and central nervous system lymphoma. Sarcomas include but are not limited to soft tissue sarcoma, osteosarcoma, malignant fibrous histiocytoma, lymphosarcoma and rhabdomyosarcoma.

In some embodiments, the cancer may comprise a hematological cancer. Hematologic cancers are cancers of the blood or bone marrow. In some embodiments, the hematological (or hematogenous) cancer of the present disclosure may comprise leukemia (acute leukemia, acute lymphocytic leukemia, acute myelocytic leukemia, acute myelogenous leukemia, myeloblastic leukemia, promyeiocytic leukemia, myelomonocytic leukemia, monocytic leukemia and erythroleukemia), chronic leukemia (chronic myelocytic leukemia, chronic granulocytic leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia B cell lymphoma, plasma cell leukemia, myelodysplasia, secondary leukemia; myeloproliferative syndromes (MPS) or Burkett's lymphoma (Endemic Burkitt's lymphoma or sporadic Burkitt's lymphoma), malignant plasma cell neoplasms, BCMA+high-grade lymphoma, Kahler's disease and myelomatosis, plasma cell leukemia; plasmacytoma, B-cell prolymphocytic leukemia, hairy cell leukemia, follicular lymphoma (including follicular non-Hodgkin's lymphoma types), marginal zone lymphoma (Mucosa-Associated Lymphoid Tissue: MALT 1 MALToma, Monocytoid B cell lymphoma, splenic lymphoma with villous lymphocytes), mantle cell lymphoma; large cell lymphoma (diffuse large cell, diffuse mixed cell, immunoblastic lymphoma, primary mediastinal B cell lymphoma, angiocentric lymphoma pulmonary B cell), small lymphocytic lymphoma (SLL), precursor B-lymphoblastic lymphoma; subacute myeloid leukemia, myeloid sarcoma, chloroma, granulocytic sarcoma, acute promyelocytic leukemia, acute myelomonocytic leukemia) or other B-cell leukemia or lymphoma.

In some embodiments, a subject of the present disclosure has a plasma cell disorder such as heavy-chain disease, primary or immunocyte-associated amyloidosis, and monoclonal gammopathy of undetermined significance (MGUS).

In some embodiments, the subject of the present treatment method has a the disease or disorder. In some embodiments, the disease or disorder may comprise acquired immune deficiency syndrome (AIDS), brain disease, acute flaccid myelitis (AFM), amyotrophic lateral sclerosis (ALS), Alzheimer's disease, amyotrophic lateral sclerosis, arthritis, bone diseases, inflammatory diseases, osteoarthritis (OA), rheumatoid arthritis (RA), asthma, blood disorders, brain disease, dementia, diabetes, enteric disease, liver disease, renal disorders lung disease, skin disease, gastrointestinal disease, ulcerative colitis, inflammatory bowel disease, hypertension, or cardiovascular disorders. In other embodiments, the disease or condition may comprise a disease or disorder of the breasts, respiratory tracts, brains, reproductive organs, alimentary canals, urethrae, eyes, livers, skins, heads and necks, thyroid glands and parathyroid glands.

In some embodiments, a subject of the present treatment method has an immunologic disorder and in particular autoimmune disorders In some embodiments, the autoimmune disorder includes, but is not limited to, systemic lupus erythematosus, myasthenia gravis, autoimmune hemolytic anemia, idiopathic thrombocytopenia purpura, anti-phospholipid syndrome, Chaga's disease, Grave's disease, Wegener's Granulomatosis, Poly-arteritis Nodosa, Rapidly Progressive Glomerulonephritis, rheumatoid arthritis, systemic lupus E (SLE), Type I diabetes, asthma, atopic dermitus, allergic rhinitis, thrombocytopenia purpura, multiple sclerosis, psoriasis, Sjorgren's syndrome, Hashimoto's thyroiditis, Grave's disease, primary biliary cirrhosis, Wegener's granulomatosis, tuberculosis, and graft versus host disease immune-mediated thrombocytopenia, haemolytic anaemia, bullous pemphigoid, myasthenia gravis, Graves' disease, Addison's disease, pemphigus foliaceus, psoriasis, psoriatic arthritis, and ankylosing spondylitis.

In some embodiments, a subject of the present treatment method has an inflammatory disease. In some embodiments, the inflammatory disease is selected from the group consisting of rheumatoid arthritis, psoriasis, allergies, asthma, autoimmune diseases such as Crohn's, IBD, fibromyalga, mastocytosis, Celiac disease, and any combination thereof. Additionally, the present method may be useful to treat diabetes, particularly Type 1 diabetes.

### Embodiments

This invention provides the following non-limiting embodiments.

In one set of embodiments, provided are:
1. A method of detecting a molecule of B cell maturation antigen (“BCMA”) in a formalin-fixed paraffin-embedded (“FFPE”) sample, comprising:
a. sectioning and mounting of the FFPE samples;
b. deparaffinizing the samples;
c. heat-mediated antigen retrieval of the samples;
d. pretreatment of the samples with blocking agents;
e. contacting the sample with a first detection agent, wherein optionally the first detection agent comprises an antibody or antigen binding fragment thereof, that binds at least one molecule of BCMA in the sample;
f. optionally, removing unbound sample;
g. contacting the sample bound to the first detection agent with a second detection agent, wherein optionally the second detection agent is an antibody or fragment thereof;
h. optionally removing the unbound second detection agent; and
i. detecting the presence of the second detection agent bound to the sample;
wherein detection above background of an amount of the second detection agent bound to the sample indicates the presence of at least one molecule of BCMA in the sample.

2. The method of embodiment 1, wherein the heat-mediated antigen retrieval is performed using an EDTA-based solution.

3. The method of embodiment 2, wherein the EDTA-based solution has a pH between 8.5 to 9.5.

4. The method of embodiment 3, wherein the EDTA-based solution has a pH of 9.0.

5. The method of any of embodiments 1 to 4, wherein the step of heat-mediated antigen retrieval is performed for 5 minutes, 10 minutes, 15 minutes, 20 minutes, 25 minutes, 30 minutes or 60 minutes.

6. The method of any of embodiments 1 to 5, wherein the step of heat-mediated antigen retrieval is performed at a temperature between 85° C. and 100° C. 7. The method of embodiment 6, wherein the step of heat-mediated antigen retrieval is performed at 100° C. 8. The method of any of embodiments 1 to 7, wherein the blocking agent blocks endogenous peroxidase.

9. The method of embodiment 8, wherein the blocking agent is a peroxide block.

10. The method of embodiment 9, wherein the step of pretreating with peroxide block is performed for 2 minutes, 5 minutes, 8 minutes, 10 minutes or 15 minutes.

11. The method of any of embodiments 1 to 10, wherein the blocking agent blocks non-specific antibody binding.

12. The method of embodiment 11, wherein the blocking agent is a Dako/Agilent Protein Block.

13. The method of embodiment 12, wherein the step of pretreating with Dako/Agilent Protein Block is performed for 5 minutes, 10 minutes, 15 minutes, 20 minutes or 30 minutes.

14. The method of any one of embodiments 1 to 13, wherein the first detection agent is an antibody or antigen binding fragment thereof.

15. The method of any one of embodiments 1 to 14, wherein the sample was contacted with the first detection agent at room temperature or at 37° C.

16. The method of any one of embodiments 1 to 15, wherein the sample was contacted with the first detection agent for 15 minutes, 30 minutes, 45 minutes, 60 minutes, 95 minutes, or 120 minutes.

17. The method of any one of embodiments 1 to 16, wherein the second detection agent is an antibody or antigen binding fragment thereof.

18. The method of any one of embodiments 1 to 17, wherein the sample bound to the first detection agent was contacted with the second detection agent for 5 minutes, 10 minutes, 15 minutes, 20 minutes, 25 minutes, or 30 minutes.

19. The method of any one of embodiments 1 to 18, wherein the sample comprises cells from a bodily fluid or tissue.

20. The method of embodiment 19, wherein the tissue is brain tissue.

21. The method of embodiment 20, wherein the tissue is from striatum, thalamus, midbrain, or medulla regions of the brain.

22. The method of embodiment 19, wherein the tissue is tumor tissue.

23. The method of any one of embodiments 1 to 22, wherein the sample is from a subject.

24. The method of embodiment 23, wherein the subject is a mammal.

25. The method of embodiment 24, wherein the mammal is a human.

26. The method of embodiment 24, wherein the mammal is Macaca fascicularis.

27. A kit for performing the method of any one of embodiments 1 to 26.

28. A method of treating a subject with a BCMA targeting agent, comprising a step of detecting BCMA in a sample from the subject.

29. The method of embodiment 28, wherein the step of detecting BCMA in the sample comprises the method of any of embodiments 1 to 22 or 24 to 26.

30. The method of embodiments 28 or 29, wherein the targeting agent is a chimeric antigen receptor (CAR) T-cell.

31. The method of embodiments 28 or 29, wherein the targeting agent is a T cell redirecting antibody.

32. The method of any of embodiments 28 to 31, wherein the subject has cancer.

33. The method of embodiment 32, wherein the cancer is multiple myeloma.

Particular embodiments of this invention are described herein. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. A number of embodiments of the invention have been described. Nevertheless, it will be understood that various modifications may be made without departing from the spirit and scope of the invention. Accordingly, the descriptions in the Examples section are intended to illustrate but not limit the scope of invention described in the claims.

### EXAMPLES

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present disclosure. Efforts have been made to ensure accuracy with respect to numbers used (e.g., amounts, percentages, etc.), but some experimental errors and deviations should be accounted for.

### Example 1: Immunochemistry Method Development

The antibodies screened are shown in Table 1.

TABLE 1
Antibodies Screened.

| | Cell Signaling | | | |
|---|---|---|---|---|
| Vendor | Technology | R&D Systems | BioLegend | Novus Biologicals |
| Clone name | E6D7B | 1004023 | Clone 19F2 | Vicky-1 |
| Isotype | Rabbit | Mouse monoclonal | Mouse | Rat monoclonal IgG1 |
| | monoclonal IgG | IgG2b | monoclonal | |
| | | | IgG2a with | |
| | | | kappa light | |
| | | | chain | |
| Immunogen | Synthetic peptide | No information | Recombinant | Synthetic peptide: |
| | including amino | | IgG Fc- | amino acid residues 2- |
| | acid residues | | BCMA | 54 (extracellular |
| | around leucine | | fusion protein | domain) |
| | 115 (cytoplasmic | | | |
| | domain) | | | |
| Applications | IHC, WB, IP, FC | ELISA, FC, | FC | IHC, IF, ICC, WB, |
| (given by each | | IHC, CyTOF | | ELISA, FC |
| providers) | | | | |
| Concentrations | 7.8 ug/mL | 10 to 2 ug/mL | 5 to 1 ug/mL | 10 to 2 ug/mL |
| used in IHC assay | (optimal IHC | | | |
| screening | concentration) | | | |
| Resulting IHC | Generated a | Generated | Generated | Generated unacceptable |
| staining in | specific and | unacceptable | unacceptable | sensitivity and |
| positive and | sensitive IHC | sensitivity and | sensitivity and | specificity (H929 |
| negative controls | staining toward | specificity (H929 | specificity | presented positivity on |
| | BCMA expressed | presented a low- | (H929 | dead cells only, U937 |
| | by the cells | intensity brown | presented | and K562 that should |
| | | wash, U937 that | positivity on | be negative presented |
| | | should be negative | dead cells | positive staining) |
| | | presented positive | only) | |
| | | staining) | | |

Generation of assay controls. Seven cell lines were selected according to endogenous BCMA RNA expression levels, based on RNA-seq data from the publicly available CCLE (https://sites.broadinstitute.org/ccle/) and Genentech (https://ega-archive.org/dacs/EGAC00001000055) data sets. The selected cell lines served as qualified reagent controls with BCMA expression at negative, low, medium, and high levels (Table 1). Since BCMA is part of a larger family of tumor necrosis factor receptors, including BAFFR and TACI, which share ligands with BCMA, additional work was done to confirm lack of antibody cross-reactivity to potentially conserved epitopes. Engineered cell lines expressing TACI or BAFFR were generated by transfection of HEK293 parental cells with lentiviruses carrying the human TACI or the human BAFFR gene coupled with a Tag epitope. Cell lines were cultured to 100% confluence, non-enzymatically collected, and pelleted by centrifugation. Cell pellets were fixed in 10% neutral-buffered formalin for 24 hours, routinely processed to paraffin, and embedded to form an array in a single paraffin block.

FFPE samples of colon, spleen, and lymph node were acquired as endogenous tissue controls due to the presence of resident BCMA-expressing plasma cells. The same tissues from cynomolgus monkey were used to confirm cross-reactivity of the antibody with this species. All tissues were quality controlled (QCed) for their anatomical location, lack of histopathology, and suitability for IHC and ISH, and only samples that met QC criteria were used in experiments. To be considered suitable for IHC, the sample had to show the expected immunolabeling pattern by synaptophysin IHC. Suitability for ISH was confirmed by abundant positive signal for the mRNA housekeeping gene, peptidylprolyl isomerase B (PPIB), and a lack of DapB (negative control probe) signal.

Screening candidate IHC reagents. The IHC assay was developed on the Leica Bond Rx autostainer (Leica Biosystems, Buffalo Grove, IL). Table 1 summarizes the IHC candidate reagents considered to develop the definitive BCMA IHC assays. The IHC reagents were tested on the cell pellet reagent controls to assess their specificity and sensitivity toward the BCMA protein. Of all reagents tested, only the rabbit monoclonal anti-BCMA antibody clone [E6D7B] presented acceptable IHC binding specificity and sensitivity. Therefore, the anti-BCMA antibody clone [E6D7B] was used in a refined assay as primary antibody for the detection of endogenous expression of BCMA protein on tissue sections.

Assay conditions. The final IHC method used rabbit monoclonal anti-BCMA antibody clone [E6D7B] (Cell Signaling Technology #88183). FFPE blocks were sectioned at 4 μm, and the samples mounted on SuperFrost Plus glass slide (VWR, cat #48311-703). Briefly, unbaked glass slides were loaded in the autostainer and were deparaffinized following the generic Leica deparaffinization protocol. Heat-mediated antigen retrieval with an EDTA-based solution (pH˜9.0) was performed at 100° C. for 20 minutes. Slides were then pretreated with endogenous peroxidase solution for 10 minutes. Dako serum-free protein block (Agilent cat #X0909) was applied on the samples for 10 minutes just before a 30 minute incubation with a 1/200 dilution of the rabbit monoclonal anti-BCMA antibody clone [E6D7B] (final concentration 7.8 μg/mL in antibody diluent [Diagnostic Biosystem #K0004]). After extensive washing steps, the bonded primary antibody was detected by the chromogenic Leica refine DAB detection kit (Leica cat #DS9800) according to the manufacturer's recommendations. The slides were mounted with glass coverslips and were examined with a bright field microscope.

Investigational human brain tissues for IHC and ISH. FFPE human normal brain tissues were commercially sourced from various vendors. Each brain tissue sample was checked for location accuracy by H&E and IHC suitability by synaptophysin staining (rabbit monoclonal anti-synaptophysin antibody 1/8000 [Abcam #32127]). A total of 107 brain samples spanning a wide range of brain loci were used in this study. The samples originated from 63 different donors. A subset of samples was further evaluated for ISH suitability using PPIB (positive control probe).

FFPE cynomolgus normal brain tissues originating from two non-sexually mature males and two non-sexually mature females were assessed from coronal sections following the species-specific brain trimming scheme. Cynomolgus monkey tissue was included as the species is routinely studied in preclinical safety of some biologic therapeutics due to the lack of biotherapeutic cross-reactivity and pharmacologic activity in other species. Standardized tissue sampling and processing protocols were followed to reduce preanalytical variables and maximize tissue sample quality.

Colocalization Studies. Colocalization studies between the IHC immunoreactivity observed with E6D7B and Golgi apparatus and neurofibrillary tangle markers were performed by immunofluorescence. Assessment of the staining was completed using a Zeiss confocal microscope (Zeiss, White Plains, NY). The colocalization experiments included the following antibody probes: cis- and trans-Golgi markers, mouse monoclonal anti-TGN46 antibody 1/500 (LSBio #LS-C133654-100), mouse polyclonal anti-GOLGA2 antibody 1/100 (Abnova #H00002801-B01P), mouse monoclonal anti-GOLGA5 antibody 1/300 (NovusBio #NBP2-66875), and mouse monoclonal anti-GOLM1 antibody 1/100 (Sino Biological #13066-MM12). Each specific antibody was detected by the following secondary binders: Goat polyclonal Alexa Fluor® 488 AffiniPure F(ab′)₂ Fragment Anti-Mouse IgG (H+L) (Jackson ImmunoResearch Laboratories #115-546-146), and goat polyclonal Alexa Fluor® 594 AffiniPure F(ab′)₂ Fragment Anti-Rabbit IgG (H+L) (Jackson ImmunoResearch Laboratories #111-586-144).

Bielschowsky Silver Stain. To highlight neurofibrillary tangles, key tissue samples were stained with the Bielschowsky silver stain method (Abcam ab245877). The method was executed according to the manufacturer's recommendation. Briefly, 4 μm tissue sections were deparaffinized and hydrated before incubation in a solution of silver nitrate for 15 minutes at 40° C. followed by 10 minutes in ammoniacal silver solution. The silver staining was developed in developer solution under agitation until desired coloration. The precipitated silver was fixed with 5% sodium thiosulfate for 2 minutes and the slides were dehydrated and mounted with Permount (Fisher Scientific, SP15-100) before digital capture. Detection of phosphorylated Tau protein was also performed using the tangle marker probe: mouse monoclonal anti-pTau 1/100 (RnD Systems #MAB34941-100).

### Example 2: RNA In Situ Hybridization Assay Development

Controls. Cell pellet arrays and human FFPE colon samples, as previously described in Example 1, served as reagent controls. For each ISH staining assay, the FFPE blocks were sectioned at 4 μm, and the samples mounted on SuperFrost Plus glass slides.

Reagents. The ISH assay was developed on the Leica Bond Rx autostainer (Leica Biosystems, Buffalo Grove, IL) using the following key reagents: the mRNA detection probes including human BCMA-specific probe (Hs-TNFRSF17, ACDBio cat #585791), human-positive tissue PPIB control probe (Hs-PPIB, ACDBio cat #313908), and a negative control probe DapB (ACDBio cat #312038). The hybridized probes were detected using the RNAscope® 2.5 LSx Reagent Kit-Red (ACDBio cat #322750).

Assay Conditions. The BCMA ISH assay was performed on the Leica Bond Rx autostainer. Briefly, glass slides were loaded in the autostainer, baked at 60° C. for 30 minutes and deparaffinized following the generic Leica deparaffinization protocol. Heat-mediated antigen retrieval with an EDTA-based solution (pH˜9.0) was performed for 15 minutes at 85° C. for the cell pellet reagent controls or 95° C. for the tissue samples. Non-specific enzymatic digestion with proteinase K (provided in the ACDBio RNAscope® 2.5 LSx kit) was then applied on each histological sample for 15 minutes at 40° C. Hybridization of the specific probes occurred at 42° C. for 120 minutes. After extensive washes, the specifically bonded probes were detected by a series of signal amplification steps. Finally, an alkaline phosphatase enzymatic activity reacted upon a chromogen, producing a red precipitate signal visible under a bright field microscope.

The ISH staining signal results in an intracellular dot-like pattern. The number of positive dots per cell generally correlates to the amount of detectable mRNA transcripts present. The size of each dot depends preferentially on the overall probe sets design. Evaluation of the ISH signal was visually performed according to the manufacturer guidelines. The minimum number of dots when considering a cell positive and its relationship to correlative protein expression depends on each target of interest. Since there can be differences between the level of mRNA and protein, a side-by-side characterization between ISH signal level and IHC immunoreactivity intensity provides acceptable indication of the ISH signal threshold to consider relative to protein expression.

Preanalytical variables in tissue harvesting and processing, such as prolonged time in ethanol, may influence the quality of mRNA that can be detected with the ISH method. The PPIB is a housekeeping gene and its ISH signal was used to assess overall quality of the mRNA present in the FFPE samples (i.e., QC check). Each cell pellet included in the control array presented an ISH PPIB signal well above the manufacturer's minimum recommended threshold of three to four dots per cell.

GTEx RNA-seq Data. RNA-seq data of the GTEx Analysis Release V8 was obtained from the dbGaP database (https://www.ncbi.nlm.nih.gov/projects/gap/cgi-bin/study.cgi?study_id=phs000424.v8.p2) and was processed using the Omicsoft Array Suite tool (https://www.arrayserver.com/wiki/). Human genome build GRCh38 and gene model GENCODE Release 33 (https://www.gencodegenes.org/human/) were adopted for mapping RNA-seq sequences to genome and quantifying gene expression. All 2641 samples of 13 brain regions, including 246 caudate samples and 204 putamen samples, were selected for the profiling of BCMA expression in human brain. Transcripts per million (“TPM”) was adopted as the unit of gene expression measurement.

Allen BrainSpan RNA-seq Data. All gene expression data were downloaded from the brain Allen BrainSpan web site (https://www.brainspan.org/) using the link https://www.brainspan.org/api/v2/well_known_file_download/267666525. Data were converted from reads per million kilobases to TPM to enable compatibility with the GTEx data. BCMA expression was reported with samples grouped by brain region and development stage.

Aggregated RNA-seq Data. To determine how expression of BCMA gene in human striatum varies with stage of development, BCMA gene expression data in striatum samples from the Allen BrainSpan dataset and in caudate and putamen samples from the GTEx dataset were extracted. The combined data were plotted together and split by age group of the donor.

### Example 3: Results of IHC and ISH Assays

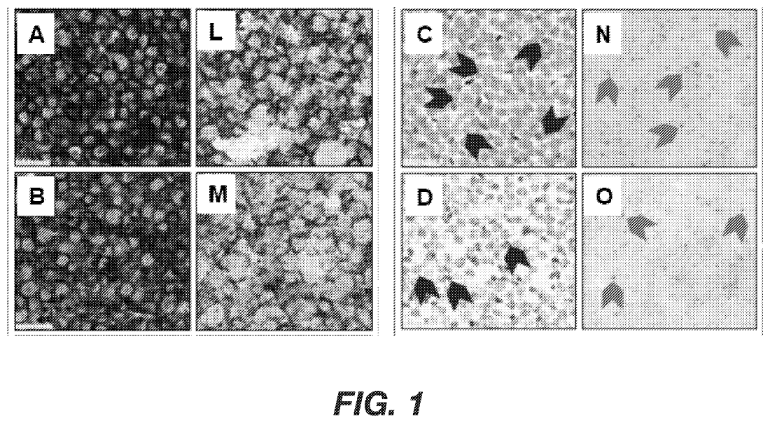

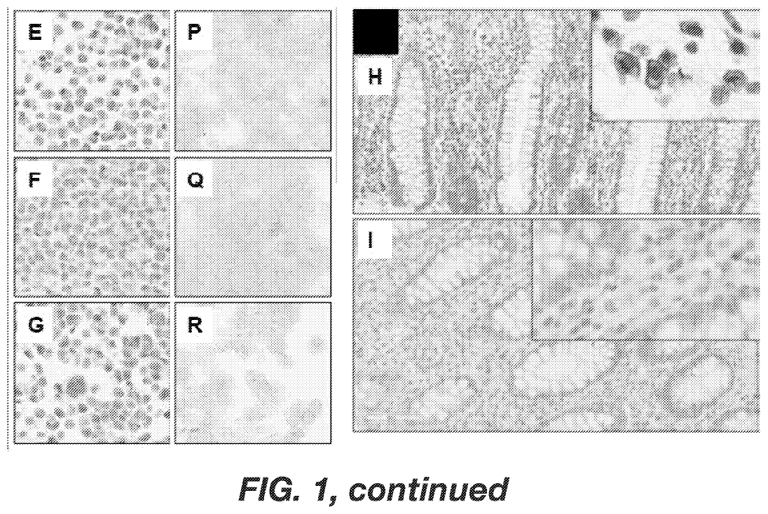

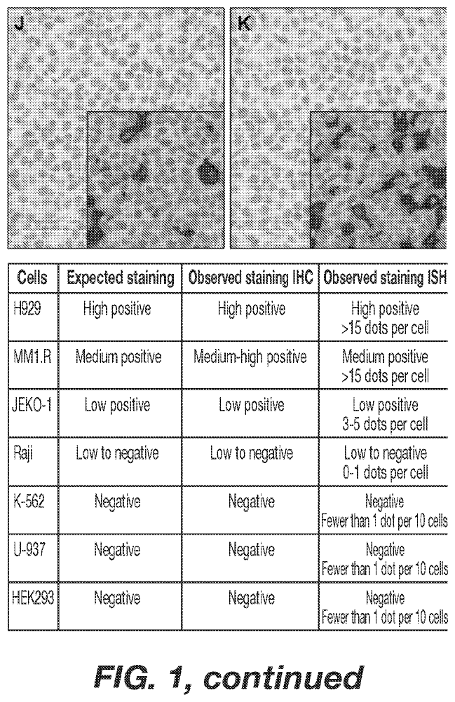

IHC Assay. The cell pellet reagent control array staining results are summarized in Table 1 and FIGS. 1A-1R. The mouse monoclonal anti-BCMA antibody clone [1004023] (RnD Systems, Cat #MAB1931), the mouse monoclonal anti-BCMA antibody clone [Clone 19F2] (BioLegend, Cat #357502), and the rat monoclonal anti-BCMA antibody clone [Vicky-1] (Novus Biologicals, Cat #NBP1-97637SS) did not generate acceptable specificity and sensitivity toward BCMA protein expressed nor by the control cell pellets (data not shown). The rabbit monoclonal anti-BCMA antibody clone [E6D7B] generated the expected pattern of BCMA-specific immunoreactivity (i.e., membrane and/or Golgi-like pattern) in cell pellet controls (FIGS. 1A-1R). H929 and MM1.R cells presented marked positive-membrane staining and a marked Golgi-like staining pattern in their cytoplasm. The JEKO-1 and Raji cells displayed a low- to very low-intensity staining, mostly observable in the Golgi-like structure present in the cells' cytoplasm. No immunoreactivity was observed with E6D7B in the BCMA-negative cell lines (K562, U937 and HEK293), as expected. BCMA immunoreactivity generated by the clone [E6D7B] was orthogonally confirmed with an ISH assay specific for detecting BCMA mRNA. HEK293-BAFFr and HEK293-TACI cell lines overexpressing the closely related targets BAFFR and transmembrane activator and CAML interactor (TACI) did not present any BCMA immunoreactivity, further indicating specificity of E6D7B to BCMA.

In tissue sections, specific positive labeling was identified in cells with morphology consistent with plasma cells in human lymph node, spleen, and colon samples. Like in the BCMA-positive cell pellet reagent controls, the plasma cells presented a strong membrane staining and a Golgi-like positive structure when present in the plane of section. The relative intensity of the positive cells in the lymph node and spleen was less than that observed in the colon tissue samples. Evidence of cross-reactivity with cynomolgus monkey tissue samples was identified in similar anatomical locations. The overall intensity of the positive BCMA IHC cells was less in cynomolgus monkey than that observed in human. The cynomolgus monkey colon presented a higher density of BCMA-positive cells than the human colon. The exact peptide immunogen sequence that was used to generate the rabbit monoclonal anti-BCMA antibody clone [E6D7B] is proprietary to Cell Signaling Technology (CST). CST published that the monoclonal antibody is produced by immunizing animals with a synthetic peptide corresponding to residues surrounding Leu115 of human TNFRSF17/BCMA protein. A bioinformatic alignment of the cytoplasmic moiety of the human and cynomolgus BCMA protein (amino acid residues 78-184) showed a 92.5% sequence identity between cynomolgus monkey and human proteins (BLAST-P, NIH).

ISH Assay. BCMA-specific ISH staining was identified in cell lines presenting a range of endogenous expression, from low to high BCMA mRNA fragments per kilobase million (FPKM) scores (Table 1, FIGS. 1A-1R). Cell lines with high BCMA FPKM scores (H929 and MM1.R) presented 15 or more dots per cell. In cells with lower FPKM scores (JEKO-1 and Raji), 5 dots or 1 dot per cell were observed, respectively. As expected, the BCMA ISH assay was not able to generate specific signal in cell lines displaying extremely low or zero FPKM scores (K562, U937, and HEK293).

The direct correlation between the BCMA ISH signal and BCMA IHC immunoreactivity in the same control cell pellets was visually estimated (Table 1, FIGS. 1A-1R). Acceptable concordances were observed on the FFPE cell pellets between the intensity of the BCMA IHC immunoreactivity and the BCMA ISH signal. Not all Raji cells displayed BCMA ISH positivity in the plane of section, and the positive cells presented mainly 1 dot and rarely 2 dots. For that cell line, the overall density of positive BCMA ISH signal matched that observed with the BCMA IHC assay reaching the threshold for positivity as determined by recommended scoring by the manufacturer (https://www.indicalab.com/wp-content/uploads/2018/04/MK_51_103_RNAScope_data_analysis_guide_RevB.pdf). Although occasional ISH signal was present in negative control cell lines (K562, U-937, and HEK293) the signal was infrequent and did not reach the one dot per ten cell threshold.

In human normal colon tissue sections (positive control), specific positive BCMA ISH labeling was identified in cells with morphology consistent with plasma cells. Most of those cells displayed a low level of BCMA ISH signal (1 dot/cell and occasionally 2-3 dots/cells). Groups of plasma cells exhibiting a single BCMA ISH dot signal appeared strongly stained by the IHC assay in subsequent adjacent sections, indicating that, similarly to the Raji control cells, IHC-detectable BCMA protein expression in tissue could be predicted for cells presenting a sustained ISH level as low as 1 dot/cell.

Presence of observable positive signal generated by the BCMA ISH assay required a particular overall mRNA quality threshold, as assessed by the PPIB QC ISH assay (4 dots/cell or greater). Plasma cells that presented a specific positive BCMA ISH signal generally had a minimum of four dots per cell present in the QC PPIB ISH signal. In contrast, there was no corresponding BCMA ISH signal detected in areas where plasma cells had low PPIB ISH signal lower than four dots/cell, despite strong and specific positive signal at the protein level (IHC on immediately adjacent sections).

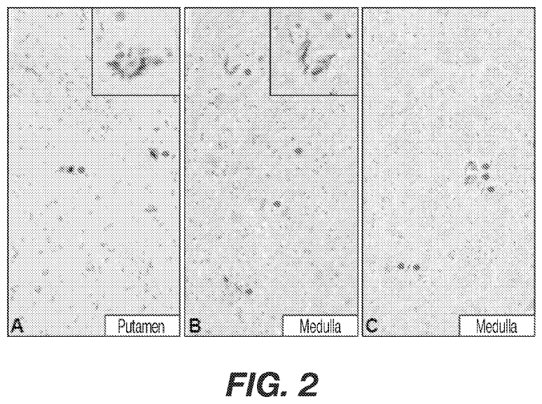

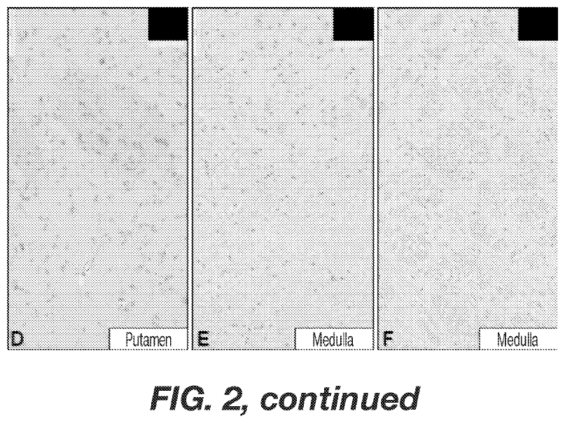

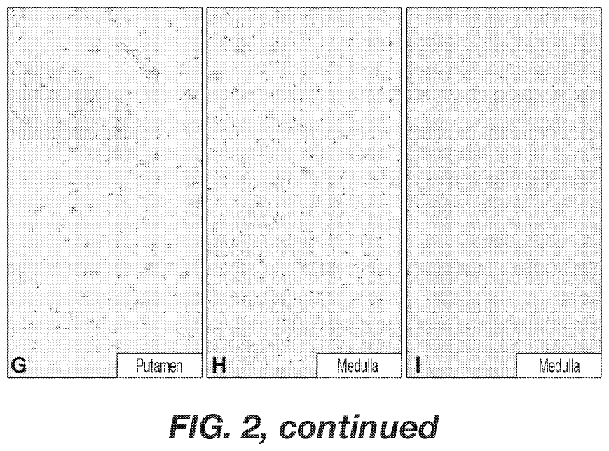

BCMA IHC on Human FFPE Normal Brain Samples. Immunoreactivity with rabbit monoclonal anti-BCMA antibody clone [E6D7B] (CST) was observed in a small subset of neurons in specific anatomical regions of several normal human brain samples. The immunoreactivity pattern presented as cytoplasmic fibril- or needle-like structures found in the cell body (soma) and axonal processes of certain neurons (FIGS. 2A-21) and rarely in glial cell processes. Neurons with IHC immunoreactivity were mainly observed in the dorsal striatum, thalamus, midbrain, and medulla oblongata. Among all neurons present on the tissue sections, the density of cells with immunoreactivity varied by the location examined and was generally low. A similar regional pattern of immunoreactivity was consistently reproduced across brain tissues and donors, regardless of demographic or commercial origin of the samples. Accounting for the relative low number of samples assessed in this study, no clear parallel was found between the IHC immunoreactivity with E6D7B and available demographic data associated with the tissue samples.

The unique fibril- or needle-like subcellular, non-membranous, non-Golgi-like pattern of immunoreactivity in human normal brain was unexpected as it greatly differed from the characteristic pattern in cells that are known to express this protein (e.g., plasma cells). The unexpected immunoreactivity warranted further characterization. To that end, orthogonal experimentations were conducted on representative human brain samples where IHC immunoreactivity was observed.

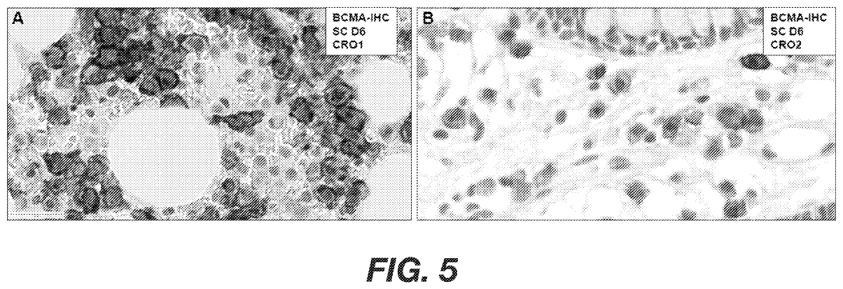

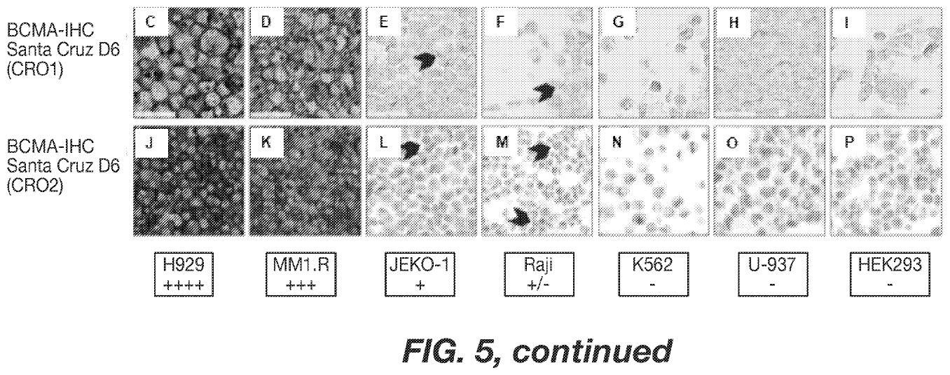

To further assess BCMA expression in the human brain samples, external molecular pathology service providers (CROs) were contracted to develop independent BCMA IHC assays, at least as sensitive as E6D7B, for application in detecting low levels of the BCMA protein. Two BCMA IHC assays developed at Reveal Biosciences (San Diego, CA) and Hematogenix (Tinley Park, IL) were based on the mouse monoclonal anti-BCMA clone D6 from Santa Cruz Biotechnology. Those two assays generated acceptable and comparable specificity and sensitivity in targeting BCMA on the same cell pellet reagent and colon tissue controls used to develop the internal BCMA IHC assay. A set of the same brain samples that demonstrated immunoreactivity using the E6D7B clone were sent to each CRO for staining in a blinded manner. In the control samples, the BCMA IHC based on the Santa Cruz anti-BCMA antibody clone D6 presented very similar staining pattern to the internal BCMA IHC (FIGS. 5A-5P). In contrast, no BCMA staining or fibril-like immunoreactivity was seen in the brain samples. Thus, a BCMA IHC assay based on the Santa Cruz mouse monoclonal anti-BCMA antibody clone D6, performed independently at two contract laboratories, did not reproduce the immunoreactivity originally observed in the brain samples with rabbit monoclonal anti-BCMA antibody clone [E6D7B].

BCMA ISH on Human FFPE Normal Brain Samples. The specific detection of mRNA transcripts from the gene of the protein of interest in the same cells and anatomical regions could increase confidence in the specificity of the observed immunoreactivity. Therefore, BCMA ISH was used to corroborate the observed immunoreactivity when using the E6D7B clone, as BCMA ISH reveals gene expression with anatomical and cellular resolution. A total of 49 brain samples, chosen at random without regard to E6D7B-mediated immunoreactivity, were stained with the mRNA QC probe PPIB. Of these 49 samples, 25 presented an acceptable PPIB control signal (4 dots per cell or more) and were used in subsequent experiments. The BCMA-specific probe was applied on those 25 samples and only four of those samples generated a very low (1-2 dots/cell) positive signal spotted in one or two cells among the thousands present in the section. A similar signal could be detected in negative control cell pellet samples (HEK293 cells), but this signal was below the threshold of what is considered positive based on the assay. The immunoreactivity produced with the E6D7B clone was seen in occasional neurons throughout the brain sections, whereas the ISH signal was exceedingly rare.

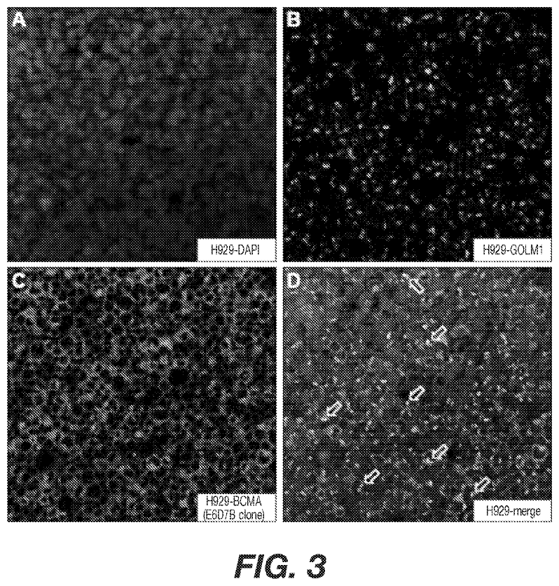

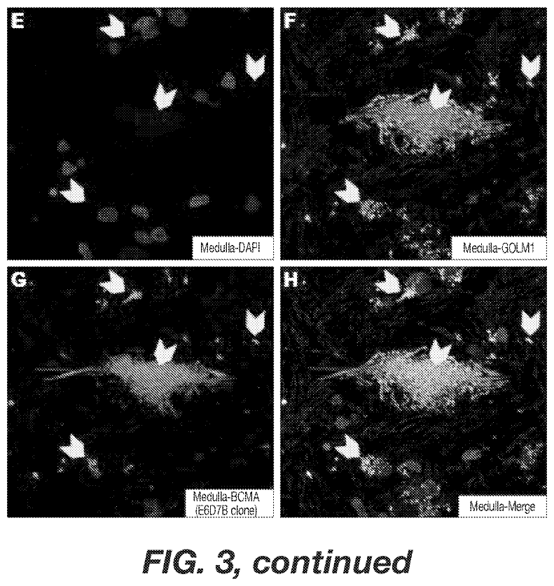

Colocalization Assays. To further characterize the immunoreactivity resulting from the BCMA IHC assay performed on normal human brain tissues, immunofluorescent colocalization experiments with Golgi apparatus markers were conducted. In plasma cells that endogenously express BCMA protein, BCMA can be found on the cell membrane or within the Golgi apparatus. The Golgi apparatus of neurons differs greatly in shape, size, and distribution when compared with plasma cells; therefore, it was deemed appropriate to determine if BCMA immunoreactivity colocalized with known Golgi proteins within the neuronal cell bodies. To cover the span of the Golgi apparatus, cis-(GOLGA2 and GOLM1, PMID: 18953438) and trans-(TGN46, PMID: 29311477) Golgi IHC markers were examined. In tissue-resident plasma cells and reagent control cell pellets, the BCMA IHC assay located the protein at the plasma membrane and invariably in a Golgi-like structure in the cell cytoplasm. As anticipated, confocal observations carried out between Golgi markers and the BCMA antibody resulted in an evident colocalization in cell pellet controls. In the neurons of the normal human brain samples, no colocalization could be observed between cis- and trans-Golgi markers and the immunoreactivity resulting from E6D7B (FIGS. 3A-3H).

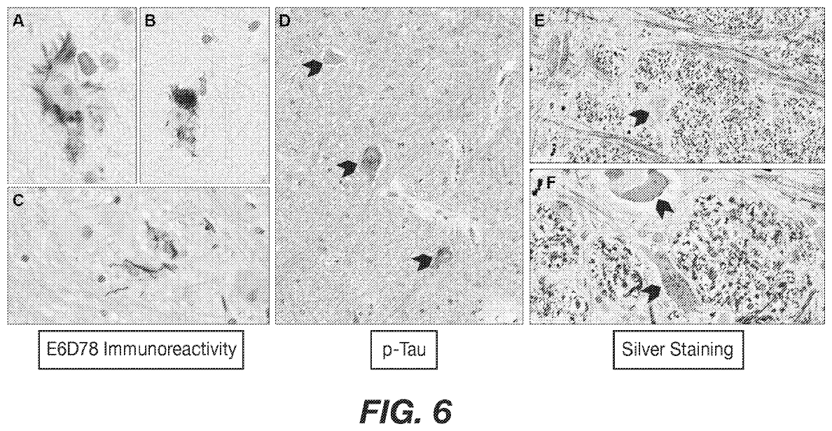

Additionally, since the immunoreactive fibrils are morphologically reminiscent of neurofibrillary tangles seen in neurodegenerative diseases, such as Alzheimer's disease, colocalization of pTau (the most common aggregating protein seen preclinically and clinically in Alzheimer's disease) with BCMA was also examined. The colocalization was assessed by confocal microscopy. The pTau IHC staining indicated absence of tangles in the tissue samples assessed; therefore, the immunoreactivity observed with E6D7B did not display any colocalization with that marker (FIGS. 6A-6F). The pTau colocalization study did not reveal any relationship with the immunoreactivity seen in the tissue samples. Since protein aggregates can be formed by a wide variety of proteins aside from pTau, a Bielschowsky silver staining was used to identify most tangles independently of the protein(s) involved. Select neurons displayed punctate intraneuronal argyrophilic aggregates and most of the cells presented a clear cytoplasm. In the medulla, the distribution and morphology of the argyrophilic aggregates did not resemble the pattern generated by E6D7B IHC, suggesting that the immunoreactivity would most likely not originate from aggregated proteins (FIGS. 6A-6F).

Comparative BCMA IHC in Cynomolgus Monkey Brain Samples. Cynomolgus macaque normal brain locations, corresponding to those considered in human, were stained with the BCMA IHC assay using the E6D7B clone. No BCMA immunoreactivity was observed in the cynomolgus tissue samples assessed, including the same anatomical locations studied in human such as caudate, putamen, and thalamus.

GTEx RNA-Seq Analysis. Across brain samples, BCMA RNA detection is generally negligible (0-0.21 TPM). In the caudate nucleus and putamen (components of the striatum) there are a small subset of samples with slightly higher TPM values (1-3 TPM).

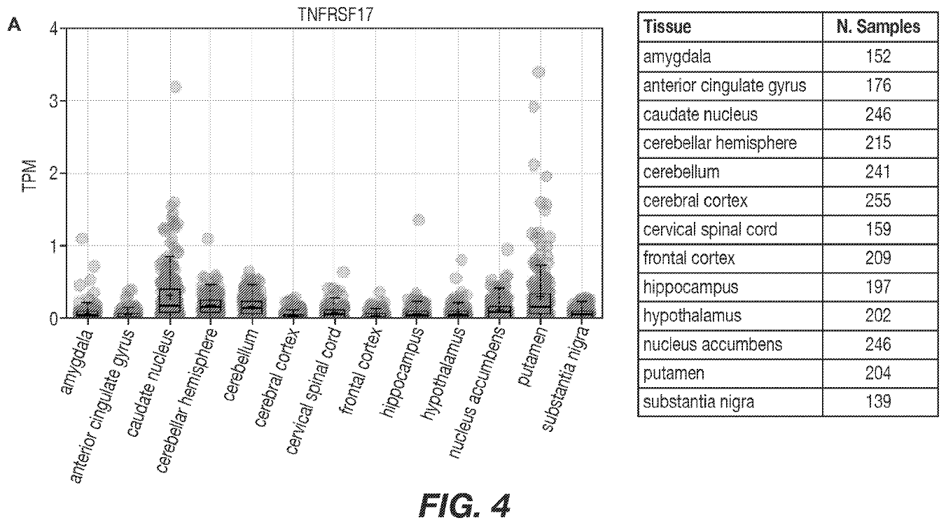

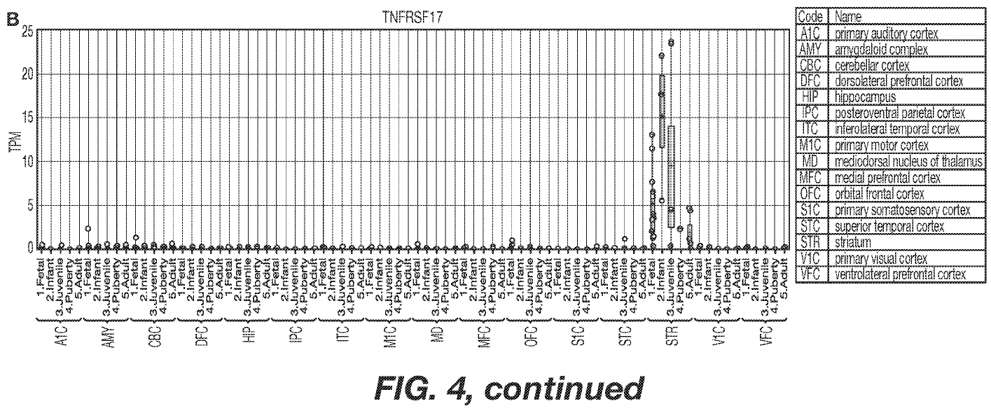

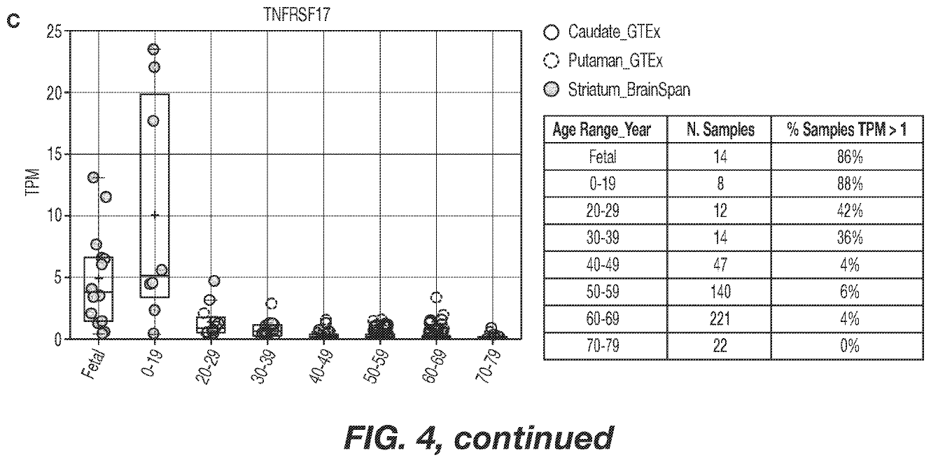

Allen BrainSpan RNA-seq Analysis. BCMA (TNFRSF17) RNA expression (FIGS. 4A-4C) is detected in the striatum in samples from fetal (average TPM=5.2), infant (average TPM=15.1), juvenile (average TPM=9.5), pubertal (one donor, TPM=2.2), and young adults (19- and 20-years old, average TPM=4.6). BCMA RNA levels are negligible (average TPM less than 0.4) in all other brain regions where multiple development stages were examined.

Aggregated BCMA RNA-seq Expression Data: Striatum. When GTEx and Allen BrainSpan BCMA RNA expression levels for the striatum and components of striatum (caudate nucleus and putamen) samples (n=478) show a clear decline in expression with increasing age (FIGS. 4A-4C). BCMA RNA expression is generally negligible in the striatum of donors over the age of 30.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. A method of detecting a molecule of B cell maturation antigen (“BCMA”) in a formalin-fixed paraffin-embedded (“FFPE”) sample, comprising:
a. sectioning and mounting of the FFPE samples;
b. deparaffinizing the samples;
c. heat-mediated antigen retrieval of the samples;
d. pretreatment of the samples with blocking agents;
e. contacting the sample with a first detection agent, wherein optionally the first detection agent comprises an antibody or antigen binding fragment thereof, that binds at least one molecule of BCMA in the sample;
f. optionally, removing unbound sample;
g. contacting the sample bound to the first detection agent with a second detection agent, wherein optionally the second detection agent is an antibody or fragment thereof;
h. optionally removing the unbound second detection agent; and
i. detecting the presence of the second detection agent bound to the sample;
wherein detection above background of an amount of the second detection agent bound to the sample indicates the presence of at least one molecule of BCMA in the sample.

2. The method of claim 1, wherein the heat-mediated antigen retrieval is performed using an EDTA-based solution.

3. The method of claim 2, wherein the EDTA-based solution has a pH between 8.5 to 9.5.

4. The method of claim 3, wherein the EDTA-based solution has a pH of 9.0.

5. The method of claim 1, wherein the step of heat-mediated antigen retrieval is performed for 5 minutes, 10 minutes, 15 minutes, 20 minutes, 25 minutes, 30 minutes or 60 minutes.

6. The method of claim 1, wherein the step of heat-mediated antigen retrieval is performed at a temperature between 85° C. and 100° C.

7. The method of claim 6, wherein the step of heat-mediated antigen retrieval is performed at 100° C.

8. The method of claim 1, wherein the blocking agent blocks endogenous peroxidase.

9. The method of claim 8, wherein the blocking agent is a peroxide block.

10. The method of claim 9, wherein the step of pretreating with peroxide block is performed for 2 minutes, 5 minutes, 8 minutes, 10 minutes or 15 minutes.

11. The method of claim 1, wherein the blocking agent blocks non-specific antibody binding.

12. The method of claim 11, wherein the blocking agent is a Dako/Agilent Protein Block.

13. The method of claim 12, wherein the step of pretreating with Dako/Agilent Protein Block is performed for 5 minutes, 10 minutes, 15 minutes, 20 minutes or 30 minutes.

14. The method of claim 1, wherein the first detection agent is an antibody or antigen binding fragment thereof.

15. The method of claim 1, wherein the sample was contacted with the first detection agent at room temperature or at 37° C.

16. The method of claim 1, wherein the sample was contacted with the first detection agent for 15 minutes, 30 minutes, 45 minutes, 60 minutes, 95 minutes, or 120 minutes.

17. The method of claim 1, wherein the second detection agent is an antibody or antigen binding fragment thereof.

18. The method of claim 1, wherein the sample bound to the first detection agent was contacted with the second detection agent for 5 minutes, 10 minutes, 15 minutes, 20 minutes, 25 minutes, or 30 minutes.

19. The method of claim 1, wherein the sample comprises cells from a bodily fluid or tissue.

20. The method of claim 19, wherein the tissue is brain tissue.

21. The method of claim 20, wherein the tissue is from striatum, thalamus, midbrain, or medulla regions of the brain.

22. The method of claim 19, wherein the tissue is tumor tissue.

23. The method of claim 1, wherein the sample is from a subject.

24. The method of claim 23, wherein the subject is a mammal.

25. The method of claim 24, wherein the mammal is a human.

26. The method of claim 24, wherein the mammal is Macaca fascicularis.

27. A kit for performing the method of claim 1.

28. A method of treating a subject with a BCMA targeting agent, comprising a step of detecting BCMA in a sample from the subject.

29. The method of claim 28, wherein the step of detecting BCMA in the sample comprises the method of claim 1.

30. The method of claim 28, wherein the targeting agent is a chimeric antigen receptor (CAR) T-cell.

31. The method of claim 28, wherein the targeting agent is a T cell redirecting antibody.

32. The method of claim 28, wherein the subject has cancer.

33. The method of claim 32, wherein the cancer is multiple myeloma.
